(19) United States
(12) Patent Application Publication
(10) Pub. No.: US 2011/0105434 A1
(43) Pub. Date: May 5, 2011

(54) 2-AMINO PURINE DERIVATIVES AND THEIR USE AS ANTI-HERPETIC AGENTS

(76) Inventor: Ray W. Exley, Beverly Hills, CA (US)

(21) Appl. No.: 12/743,535

(22) PCT Filed: Nov. 20, 2008
(86) PCT No.: PCT/US08/84246

§ 371 Date: Dec. 27, 2010

Related U.S. Application Data
(60) Provisional application No. 60/989,794, filed on Nov. 21, 2007.
(60) Provisional application No. 60/989,793, filed on Nov. 21, 2007.
(60) Provisional application No. 60/989,792, filed on Nov. 21, 2007.
(60) Provisional application No. 60/989,789, filed on Nov. 21, 2007.

(51) Int. Cl. A61K 31/52; C07D 473/18; C07D 473/34; C07F 9/6561; A61K 31/522; A61P 37/02; A61P 25/00; A61P 31/22; A61P 21/02; A61P 21/00; A61P 29/00; A61P 13/12; A61K 31/675

(52) U.S. Cl. 514/81; 544/276; 544/277; 544/244; 514/263.38

(57) ABSTRACT

Anti-herpetic material such as 2-amino purine derivatives to prevent or treat autoimmune disease or a disease originating from an abnormal functioning of the sympathetic chain in a human subject. The disease is often of a tissue or organ associated with decreased blood flow to the tissue or organ (correlated with hypertonicity of the vessels feeding the tissue or organ). The treatment reduces inflammation, scarring, destruction, and pain in the tissue or organ affected and returns the tissue or organ to nearly normal functioning, while showing none of the side effects of previous treatments. Prolonged use of the anti-herpetic compounds reduces prodrome, vesicle formation and viral shedding. The anti-herpetic compounds may be administered alone or in combination with a compound that reduces the rate of renal excretion of the anti-herpetic compound. The anti-herpetic compounds are particularly useful when administered at a level equivalent in activity to 250 mg/kg famciclovir per day.

### CROSS-REFERENCE TO RELATED PATENT APPLICATIONS

This application claims priority to provisional patent applications U.S. Ser. Nos. 60/989,789, 60/989,792, 60/989,793 and 60/989,794, all filed on Nov. 21, 2007, each of which is incorporated herein by reference in its entirety.

### FIELD OF THE INVENTION

The present invention relates to the use of anti-herpetic material such as 2-amino purine derivatives to prevent or treat autoimmune disease or a disease originating from an abnormal functioning of the sympathetic chain in a human subject. The disease is often of a tissue or organ associated with decreased blood flow to the tissue or organ (correlated with hypertonicity of the vessels feeding the tissue or organ). The treatment reduces inflammation, scarring, destruction, and pain in the tissue or organ affected and returns the tissue or organ to nearly normal functioning, while not showing any of the side effects of previous treatments. The anti-herpetic compounds may be administered alone or in combination with a compound that reduces the rate of renal excretion of the anti-herpetic compound.

### BACKGROUND OF THE INVENTION

It is known in the prior literature that certain compounds are useful for treating viruses in the herpes simplex family, e.g., herpes simplex virus—type II (“HSV-II”) and herpes zoster virus (“HZV”). These compounds include acyclovir, famciclovir, penciclovir, and others. See U.S. Pat. Nos. 4,199,574 to Schaeffer issued 22 Apr. 1980; 5,059,604 to Krenitsky, et al. issued 22 Oct. 1991; 5,246,937 to Harnden, et al. issued 21 Sep. 1993; 5,250,688 to Harnden, et al. issued 5 Oct. 1993; and 5,075,445 to Jarvest, et al. issued 24 Dec. 1991.

The Food and Drug Administration (FDA) currently approves acyclovir and penciclovir for the treatment of vesicle outbreaks of HSV-I, HSV-II, herpes zoster (shingles), and varicella (chickenpox). Famciclovir (a pro-drug for penciclovir) is FDA approved for the treatment of the vesicle outbreak phase of herpes zoster and genital herpes (HSV-II). Such treatment requires blood levels between 0.5 ng/mL and 1.0 ng/mL for acyclovir and similar levels for penciclovir. The currently recognized oral doses required to reach this blood level for an adequate amount of time (which varies according to the virus being treated and is based upon the effective half-life of the drug) for reasonable therapeutic effect are as follows:

1. FAMVIR brand famciclovir is approved for use against herpes Zoster at the oral doses of up to 500 mg three times per day for a 100 kg person, or about 1.5 g and against HSV-II; and at doses of 125 mg t.i.d. up to 250 mg t.i.d. for suppression of recurrent genital herpes. The absorption is linear in this dose range.
2. ZOVIRAX brand acyclovir is approved for several different uses against several different presentations of herpes viruses at oral doses that range between 200 mg three times per day and 800 mg per 100 kg 5 times per day. This would amount to a high dose of 4 g per day for a 100 kg subject. Because the absorption of acyclovir is non-linear in this dose range GlaxoSmithKline, the manufacturer, has discouraged the use of higher doses because it believes little more can be absorbed with doses higher than the maximum dose of 800 mg 5 times per day, which in most patients gives a blood level of about 1.61 ng/mL.
3. DENAVIR brand of penciclovir cream is approved for the treatment of herpes labialis (cold sores or HSV-I).

U.S. Pat. No. 5,559,114 (“the '114 patent”) and International Application No. PCT/US95/16207 (“the '16207 application”) teach that the administration to patients with autoimmune disease of high doses (about 8 times the recommended dose) of the above mentioned anti-viral drugs results in improvement in the patient's condition.

### SUMMARY OF THE INVENTION

One aspect of this invention is a method for treating or preventing an autoimmune disease or a disease or condition originating from an abnormal functioning of the sympathetic nervous system in a human subject. This method of treatment comprises administering on a daily basis to the subject in need thereof a therapeutically effective amount of a compound represented by Formula (I) or Formula (II), or a pharmaceutically acceptable salt thereof, below for a period of time sufficient to alleviate the subject's signs or symptoms associated with the disease, wherein the therapeutically effective amount of the compound is equivalent in activity to at least about 250 mg famciclovir per kg body weight of the subject per day. The method is particularly useful in a subject that is infected with a herpes virus, such as HSV-II, with famciclovir being the compound.

Another aspect of this invention is a method for treatment of a subject exhibiting the signs or symptoms of a disease that include chronic pain, failure of muscles to relax, sudden muscle spasm, severe fatigue, or a loss of control of or sensation in autonomic muscle. The method comprises choosing a compound of Formula (I) or (II), or a pharmaceutically acceptable salt thereof, that is equivalent in activity against the virus to at least 250 mg of famciclovir per kg body weight of the subject per day, calculating the amount of the compound needed as therapeutically effective for the subject, administering the compound at the amount calculated for a period of time sufficient to alleviate the signs or symptoms in the subject, and continuing the administration of the compound to the subject at the calculated amount. Optionally, and preferably, the subject is first tested for the presence of a herpes virus, such as HSV-II, and if positive, the other steps are undertaken.

Another aspect of the invention is a system, i.e., a product, for treating a human subject having an autoimmune disease, or a disease originating from abnormal functioning of the subject's sympathetic nervous system, which system comprises (a) a container holding a pharmaceutical composition comprising a pharmaceutically acceptable excipient and a compound represented by Formula (I) or (II), or a pharmaceutically acceptable salt thereof, and (b) instructions associated with the container for administering the pharmaceutical composition to the subject at a therapeutically effective amount equivalent in activity to at least about 250 mg of famciclovir per kg body weight of the subject per day.

Another aspect of the invention is the use of a compound of Formula (I) or (II), or a pharmaceutically acceptable salt thereof, in the preparation of a composition for treating a human subject for a disease discussed herein, wherein the composition is administered to the subject at an amount equivalent to the activity in the subject of at least about 250 mg of famciclovir per kg body weight of the subject per day.

Another aspect of the invention is a method for treating or preventing a disease in a human subject having signs or symptoms of a disease originating from an abnormal functioning of the sympathetic nervous system or an autoimmune disease. The method comprises (a) testing the subject for the presence of a herpes simplex virus, and (b) if the test is positive, administering on a daily basis to the subject a therapeutically effective amount of a compound represented by Formula (I) or Formula (II), or a pharmaceutically acceptable salt thereof, for a period of time sufficient to alleviate signs or symptoms of the subject associated with the disease, wherein the therapeutically effective amount of the compound is equivalent in activity to at least about 250 mg famciclovir per kg body weight of the subject per day.

Another aspect of the invention is a composition for treating a human subject having a disease originating from abnormal functioning of the subject's sympathetic nervous system or an autoimmune disease, and further being infected with a herpes virus, which composition comprises (a) a compound represented by Formula (I) or (II), or a pharmaceutically acceptable salt thereof, and (b) a pharmaceutically-acceptable excipient that aids in dissolving or suspending the compound of (a) in water so that the pharmaceutical composition may be administered to the subject at a therapeutically effective amount equivalent in activity to at least about 250 mg famciclovir per kg body weight of the subject per day.

Another aspect of the invention is a liquid composition for treating a human subject having a disease originating from abnormal functioning of the subject's sympathetic nervous system or an autoimmune disease, which composition comprises (a) a compound represented by Formula (I) or (II), or a pharmaceutically acceptable salt thereof, and (b) a liquid pharmaceutically-acceptable excipient that aids in dissolving or suspending the compound of (a) so that the pharmaceutical composition may be administered to the subject at a therapeutically effective amount equivalent in activity to at least about 250 mg famciclovir per kg body weight of the subject per day.

Another aspect of this invention is a composition for treating a human subject having an autoimmune disease or a disease originating from abnormal functioning of the subject's sympathetic nervous system, particularly where the subject is infected with a herpes virus, which composition comprises (a) a compound represented by Formula (I) or (II), or a pharmaceutically acceptable salt thereof, (b) a compound that decreases the rate of renal excretion of the compound of (a), and (c) a pharmaceutically-acceptable excipient.

Another aspect of this invention is a method for improving reduced renal function as measured by creatinine clearance in a human subject having an autoimmune disease or a disease originating from abnormal functioning of the subject's sympathetic nervous system, which method comprises administering on a daily basis to the subject a therapeutically effective amount of a compound represented by Formula (I) or Formula (II), or a pharmaceutically acceptable salt thereof, below for a period of time sufficient to increase the rate of creatinine clearance in the subject, wherein the therapeutically effective amount of the compound is equivalent in activity to at least about 250 mg famciclovir per kg body weight of the subject per day.

For each of the various aspects of the invention, Formula (I) is

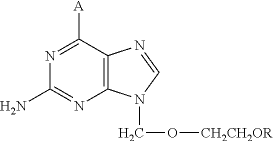

where A is H or OH and OR is OH, a lower alkyl ester of 2-4 carbon atoms, or OC(O)CH(NH₂)R₄ where R₄ is H or alkyl of 1-4 carbon atoms; and

Formula (II) is

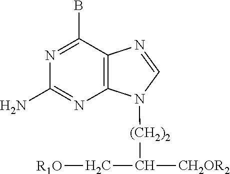

wherein B is hydrogen, chlorine, alkoxy of 1-6 carbon atoms, phenoxy, phenyl(C₁₋₆)alkyloxy, NH₂, OH or SH; each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group.

For each of the aspects of the invention the compound of Formula (I) or (II), or a pharmaceutically acceptable salt thereof, may be administered alone or in combination with a compound that decreases the rate of renal excretion of the anti-herpetic drug. An example of such a drug is probenecid.

Other aspects of the invention may be apparent to one of ordinary skill in the art upon reading the following specification and claims.

### DETAILED DESCRIPTION OF THE INVENTION

This invention is based in part on the inventor's discovery that extraordinarily high doses of certain 2-amino purine derivatives are useful for treating a broad range of diseases or conditions previously not adequately treated by any known means. The cause of these diseases was and is unknown in the general medical knowledge. This invention is also based in part on the inventor's specific discovery that autoimmune diseases and related conditions in humans can be successfully treated by administering levels of anti-herpetic compounds to a subject that are significantly higher than have previously been used in the known art and that such treatment results in significantly reduced side effects.

While not wishing to be bound by any particular theory or explanation of why the invention works in its various aspects, the mechanism of causation proposed in this application has not been previously appreciated, and/or associated, with these diseases. However, the effectiveness of this treatment establishes a cause that is linked to the activity of one or more herpes viruses in the body, and the body's response to that virus, particularly the immune system response.

The current state of knowledge before the work described in this application, has resulted in many of these conditions having many different names, most of which occur as or associated with “autoimmune” diseases, which in current knowledge means the cause is unknown. Such conditions include those with particularly severe chronic pain including fibromyalgia or irritable bowel syndrome, and many other diseases and conditions referred to by other names discussed hereinafter. Based on evidence available, the inventor believes that the cause of all of these diseases is the direct effect of the virus and the body's response to the presence of a herpes virus, particularly HSV-II, genital herpes. It is thought that this group of signs or symptoms of the diseases discussed herein is mediated by the herpetic irritation of the sympathetic nervous system, which acts on the smooth muscle cells of various organs.

The sympathetic division of the autonomous nervous system controls the tonus of involuntary and voluntary muscles, such as, for example, smooth muscles of the arterial walls and the intestinal walls as well as the resting tone of skeletal muscles. The nerve cells of the sympathetic nervous system are a preferred host of HSV-II. The hypothesis of the inventor is that as a result of the persistent viral infection and shedding of the viral particles or proteins in the sympathetic nervous system, these neurons are under constant attack from the immune system. These infected nerves fire at an increased rate as a result of (1) direct irritation of the cells by the virus, or (2) the autoimmune attack on the cells of the sympathetic chain in which the virus reproduces, or (3) both of (1) and (2).

A model for this would be the behavior of HSV-I. Shingles is caused by a persistent HSV-I infection that manifests itself years after the original infection. It has been documented that this infection causes sensitization to touch and constant firing of sensory neurons, resulting in severe pain that often continues for months or years in the location previously occupied by the shingles vesicles, long after they have disappeared. It is likely that other neurons, including pain neurons in the sympathetic chain and neurons that control tonus, are similarly irritated and activated, resulting in pain and hypertonus. HSV-II also can cause diminished or stopped firing of the cells, shutting down some functions and sensations. HSV-II prefers the cells of the sympathetic chain, which it will inhabit first after a human acquires this virus. Later it may infect other cells of the nervous system.

The inventor in this application is aware of the prior art teachings of U.S. Pat. No. 5,559,114 (“'114”) and International Application No. PCT/US95/16207 (“'16207”) that describe the treatment of certain autoimmune disease using higher than currently FDA-approved amounts of certain 2-amino purine derivatives. According to the teachings of the '114 patent and the '16207 application, one compound useful was acyclovir (or its precursor valaciclovir) at a level of 20 to 50 g per day for a 100 kg subject (or 5-10 g of valaciclovir). Another compound was famciclovir at about 4-10 g per day for a 100 kg subject (up to 12 g/kg/day). In following the treatment regimen so taught for some patients having HSV-II infection and autoimmune disease signs or symptoms, the inventor of the present invention observed that although the signs or symptoms of autoimmune disease improved, the patients continued to experience discomfort even if the high dose regimen of famciclovir was followed for a number of years. While the patients indicated significant, subjective improvement compared to their conditions prior to beginning treatment in accordance with the general teachings of the '114 and '16207 documents, they were not returned fully to normal operations. Nearly ten years after the issuance of the '114 patent and ongoing treatment as taught in that patent, the patient presented with the onset of very severe aseptic meningioencephalitis (“AME”), which was excruciatingly painful. Thus, it was that the treatment disclosed in the '114 patent was not adequate to suppress AME in the patient.

AME is a meningeal reaction (i.e., inflammation) in the cerebrospinal fluid sometimes occurring in the absence of an infection organism, but can be caused by a virus, foreign substance, diagnostic or therapeutic procedure, or a tumor or a septic focus within the skull or spinal canal. Meningitis of viral origin can be due to a picorna virus, toga virus, herpes virus, paramyxo virus, or arena virus infection. The cause of AME in this patient was not known, but suspected by this inventor to be caused by a herpes virus.

The inventor thought that the cause of the AME was the patient's herpes infection that had spread internally to the brain and meninges. The inventor found that continued treatment using the maximum dose of 120 mg/kg body weight as taught in the prior art (i.e., '114) was ineffective in treating the AME. Because the product inserts for the anti-viral products on the market (e.g., ZOVIRAX®, VALTREX®, FAMVIR®) all warned against using increased doses of the product because of the fear of further renal damage, there was reason not to use increased doses beyond those of '114. Nonetheless, the inventor tried increasing the dose of famciclovir much beyond those of '114 and discovered that by significantly increasing the doses of famciclovir to the subject to more than 250, preferably more than 300 mg/kg body weight/day, e.g., 340 to 400 mg/kg/day, the acute pain and the AME was completely stopped. This was not anticipated, but, it was also discovered that other signs or symptoms of the subject's autoimmune disease were significantly improved upon sustained high dose delivery to the subject, compared to the doses of '114.

Particularly unexpected was the significant improvement in renal function, as measured by creatinine clearance level. It had improved on the doses of '114 from 29 mL/min to 89 mL/min and had been stable for nearly ten years with those doses. However, when the dose was increased from the level taught in '114 to the levels of this application the creatinine clearance level nearly doubled from 89 mL/min to 143 mL/min. Considering the manufacturers' warnings that higher doses could, and probably would, be renal toxic, the marked improvement in renal function (almost doubling CrCl), even over the renal improvement obtained with the treatments taught in the '114 patent was unanticipated. However, careful testing of measured creatinine clearance and serum creatinine levels are both consistent with each other and both demonstrate the further improvement in renal function described herein.

This was unusual in light of the prevailing opinion in the field (at the time of filing the '114 patent and still today) that anti-herpes drugs are not helpful, but dangerous for patients with impaired kidney function. The drug makers themselves (Novartis for FAMVIR® and GlaxoSmithKline for ZOVIRAX® and VALTREX®) teach reducing the amount of the drug in proportion to kidney function as measured by creatinine clearance. For example, a person with clearance that is 50% of normal, should get 50% of the Famvir® dose for a genital herpes outbreak. If the clearance is at 25% of normal, the patient gets a 25% dose of Famvir®. For Zovirax®, the manufacturer recommends taking the same dose, but at longer intervals. A person with 50% kidney function would take the drug every 8 hours instead of every 4 hours. For Valtrex®, both the reduction in dose and lengthening the time interval are recommended.

These discoveries lead to one aspect of this invention, which is a method for treating an autoimmune disease in a human subject having such disease by using very high doses of an anti-viral drug, much higher than in '114. The method comprises administering on a daily basis to the subject a compound represented by Formula (I) or Formula (II) below, wherein the daily dose of the compound is equivalent in its effect on the subject to at least 250 mg famciclovir per kg body weight of the subject per day, preferably at least 300 mg/kg per day. The dose is maintained in the subject over time to ensure relief to the subject that is at a level to overcome the low cell membrane solubility and increase the drug level inside the cell.

A disease treated in accordance with another aspect of this invention usually is caused by decreased blood flow to the affected tissue or organ, which in turn is caused by over-contraction of the small blood vessels supplying the tissue or organ. The over-contraction is caused by “hypertonus” of the muscles of the walls of the small blood vessels. The hypertonus is caused by the over activity of the sympathetic cells that control the tension or tonus of the resting muscle cells. The sympathetic chain over-activity is caused by the virus reproduction inside the cells of the sympathetic chain. The treatment of this aspect of the invention is thought to be effective because treatment restores the sympathetic nerves to a more normal state by suppressing the virus, which lowers or eliminates the abnormal firing of the sympathetic nerves thus leading to proper tonus of the muscle cells that affect the proper blood flow in the tissue or organ affected. The resulting restored normal blood flow reduces inflammation, scarring, destruction, and pain that was caused by the abnormal sympathetic chain activity and altered blood flow, secondary to the virus activity in the cells of the sympathetic chain. This treatment suppresses the herpes virus reproduction and returns the sympathetic chain, blood flow and the tissue or organ back to nearly normal functioning. Also, sympathetic nervous system functions previously shut down, presumably by the virus activity, recover both motor and sensory functions.

This entirely new theory of causation leads to this new treatment for these (autoimmune) diseases and other related conditions. This treatment is much more effective with fewer serious side effects than prior treatments that were only intended to suppress the immune system, but do nothing to suppress the causative agent, the virus. That was because, prior to this inventor's work, the cause of all of these diseases, including “autoimmune diseases”, and related conditions was unknown. The fact that these compounds, which have little effect in the human body but to suppress thymidine kinase, is good evidence that one or more of the herpetic viruses, particularly HSV-II, is the cause of these conditions. This treatment has few serious side effects, and none of the side effects of previous treatments of '114.

This treatment with much higher than recommended dose levels has not been previously attempted because the manufacturers of currently approved anti-viral drug products imply that the doses of this patent that are needed to suppress the virus, would be renal toxic. This inventor's work contradicts such advice. The inventor is not aware of any studies that were performed that demonstrated renal damage at the currently recommended or higher dose levels. The warning is based apparently solely upon the observation that drug crystals were observed in the kidneys of sacrificed animals after they received doses higher than currently recommended by the manufacturer of an FDA-approved product, but much lower than the doses taught in this application. This finding of crystals was apparently extrapolated by the manufacturer to mean that kidney damage would occur from the crystals. The research of this inventor has demonstrated in multiple individuals taking doses from 6× to 20× the currently recommended doses for months that no renal damage occurs, particularly if the anti-herpetic is co-administered with a compound that reduces the rate of renal excretion, e.g., probenecid.

The treatment of this invention has restored renal function previously thought to be lost to these diseases, again something that contradicts prior medical knowledge and beliefs. That is because prior knowledge asserts that renal (kidney) disease and shutdown from autoimmune diseases is caused by the immune system's direct attack upon the tissues of the kidney. The work of this inventor demonstrates that is not the primary mechanism of renal shutdown. The primary mechanism is restriction of blood flow to the small vesicles of the kidneys by sympathetic chain over activity. When the virus is suppressed by these treatments, the blood flow returns to nearer normal in the kidneys. And the kidneys again function nearer to normal. The inventor demonstrated in '114 increased measured (not estimated) a 24 hr creatinine clearance level (CrCl) of from 29 mL/min to 89 mL/min, which remained stable with the continuing treatment of '114. With the increased doses described in this application over the doses of '114, the CrCl increased from 89 mL/min to 143 mL/min. (Normal CrCl is 100 to 130 mL/min. This patient was a 100 kg man, or larger than the 80 kg normal man, which explains the high 143 mL/min CrCl.) This patient was the same patient used in '114 for the results above. Simply, the renal function nearly doubled with the increased doses of this application, compared to the doses recommended in '114. Clearly the treatment of this application is a major improvement over the treatment of '114. And, this demonstrates that prior beliefs were incorrect concerning the mechanism of renal shutdown in some, perhaps all, patients suffering that problem caused by autoimmune disease. Of course, the present invention is based on further data developed by the inventor.

As mentioned above, the '114 patent and the '16207 application teach that administration of high levels of certain 2-amino purine derivatives to patients with autoimmune disease results in certain improvements in the patient's condition. However, the inventor discovered that while the previous treatment resulted in amelioration of certain signs or symptoms to allow a patient to function in a more normal fashion, the autoimmune disease can be still be debilitating, with only moderate recovery of organ function and continued (though reduced) chronic pain. Subjectively, a patient felt noticeably better as a result of the previous treatment, even to the extent of thinking 80 or 90% of the symptoms were relieved, but clearly additional relief was desirable. Other symptoms present, such as unpredictable diarrhea and ongoing occasional severe pain. As pointed out above, the discovery of the effectiveness of the use of extraordinarily high doses of certain anti-viral compounds has led to an entirely new theory of the causes and mechanisms of a wide range of diseases and to successful, ongoing methods and systems of treatment of the newly identified organization of a family of diseases, as discussed in greater detail hereinafter.

### Discussion of Autoimmune Disease

An autoimmune disease is meant to encompass those conditions in which the body's immune system produces antibodies to the body's own tissue (i.e., endogenous antigens). Generally the conditions are associated with inflammation, tissue destruction and pain. Many conditions are included under the autoimmune disease “umbrella” and are set out in the Merck Manual of Diagnosis and Therapy, Sixteenth Edition, Robert Berkow, M. D., Editor-in-Chief, 1992, Merck Research Laboratories (the “Merck Manual”) at Chapter 20 and elsewhere. Although, much is known about these diseases, the etiology of all of them is unknown as is the relationship between the various autoimmune disease conditions. It is known that in the most severe cases there is demonstrable antibody to otherwise apparently normal body tissue (i.e., auto-antigens). However, there is a large spectrum of autoimmune-associated conditions that frequently occur in patients without demonstrable circulating auto-antibodies. These include the myofascial pain syndrome, the irritable bowel syndrome, interstitial cystitis, etc. It is not known to current medical science whether these autoimmune-associated disorders are milder expressions of the same disease processes or totally separate entities.

While it is assumed by many researchers in this field that there are causative agents for these conditions, such agents have not heretofore been identified. Needless to say, the effectiveness of the treatment of this invention implies that the cause of “autoimmune diseases” and related conditions are the effects upon the human body of one or more of the herpetic viruses in the body and the immune response to same. Perhaps they will now be renamed from “autoimmune disease” to rheumatic diseases in response to herpetic viruses. This would be similar to the nomenclature of rheumatic disease from strep infections. However, bacterial infections can be cured. Currently the treatment of this invention suppresses the virus, and this prevents the disease from manifesting. But, the patient is not cured, as they must maintain a blood level of the 2-aminopurines that is adequate to suppress the virus inside the cells of the nervous system and other places such as the joints of rheumatic arthritis. At least four possible mechanisms are recognized for developing an immune response to auto-antigens. These are briefly discussed in the Merck Manual, chap. 20. Many diseases that are thought to belong to this family of disorders are set forth at p. 340 of the Merck Manual. In the population afflicted with autoimmune disorders, subjects exhibit signs or symptoms such as autoimmune inflammation, tissue destruction and often a very high level of pain. Frequently these signs or symptoms are so debilitating that a subject is so overcome that he or she can't function normally.

Major diseases include Systemic lupus erythematosus (SLE), rheumatoid arthritis (RA) and systemic sclerosis (scleroderma, old name). The latter may occur with or without autoimmune renal destruction and autoimmune associated conditions including but not limited to irritable bowel syndrome, tic douloureux (trigeminal neuralgia), myofascial pain syndrome, interstitial cystitis. Also included is multiple sclerosis and chronic non-cancer pain. While some clinicians may consider chronic non-cancer pain as a distinct, unrelated entity, it is included here with the view that it is another manifestation of autoimmune disease, which often includes myofascial pain syndrome or trigger point syndromes as part of the manifestations of “autoimmune diseases.” This inventor believes that all of these are caused by one or more of the herpetic virus's activity inside the cells of the human body, particularly of the sympathetic chain, and the body's responses to the virus, particularly the immune system response. The inventor believes that his discoveries essentially prove that “autoimmune diseases” and the conditions that often accompany such diseases are actually causes by the herpetic virus and the body's response to same. At least the inventor has demonstrated that if doses of anti-herpetic drugs are given in quantities adequate to push these drugs that are relatively insoluble in cell membranes to adequate intracellular levels, then all the signs or symptoms of autoimmune diseases are essentially totally suppressed. The 2-aminopurines of this invention do nothing but tie up a specific enzyme, thymidine kinase. The viral version of this enzyme is necessary for herpes viruses to reproduce. Since suppression of herpes virus reproduction stops and alleviates essentially all of the symptoms of “autoimmune diseases” and their related conditions like severe non cancer chronic pain, this is nearly proof that the diseases are caused by the suppressed virus. Regardless of the underlying theory, the treatments are very effective at suppressing essentially all manifestations of autoimmune diseases and related conditions, which has been clearly demonstrated in human patients. Also, the levels of drug ingestion, which previously was believed to be safe if the amount was less than about 1500 mg/day, or 15 mg/kg/day in the 100 kg person. This inventor has demonstrated that it is safe to ingest by mouth much higher doses, in excess of 500 mg/kg/day, or doses 33 times higher than the currently recommended maximum dose. And the doses of '114 were a maximum of 120 mg/kg/day. The inventor has demonstrated that doses of greater than about 250 mg/kg/day are much better than the effect of 120 mg/kg/day, and that doses in excess of 350 mg/kg/day rarely improve the benefits, as that dose nearly always suppresses all signs or symptoms of autoimmune diseases. However, some conditions such as aseptic meningitis may require doses in the range of 400 mg/kg/day to suppress the AME.

The Sixteenth Edition of the Merck Manual at page 340, Table 20-3 organizes putative autoimmune disorders into three groups: (1) highly probable, (2) probable, and (3) possible as follows:

(1) Hashimoto's thyroiditis, SLE, Goodpasture's syndrome, pemphigus, receptor autoimmunity (e.g., Grave's disease, myasthenia gravis, insulin resistance), autoimmune hemolytic anemia, and autoimmune thrombocytopenic anemia;
(2) rheumatoid arthritis, scleroderma with anti-collagen Abs, mixed collective tissue disease, polymyositis, pernicious anemia, idiopathic Addison's disease, infertility, glomerulonephritis, bullous pemphigoid, Sjogren's syndrome, diabetes mellitus (some), and adrenergic drug resistance (some with asthma or cystic fibrosis);
(3) chronic active hepatitis, primary billiary cirrhosis, other endocrine gland failure, vitiglio, vasculitis, post-MI cardiotomy syndrome, urticaria, atopic dermatitis, asthma (some cases), and many other inflammatory, granulomatosus, degenerative, and atrophic disorders.

The method of this invention is useful for treating autoimmune conditions when they present in a subject having a herpes virus infection. As mentioned above, a subject having an autoimmune disease generally will exhibit signs and/or symptoms associated with the autoimmune disease. A sign is generally any abnormality indicative of a disease that is discoverable on examination of a subject; it is an objective indication of the disease. A symptom, on the other hand, is any departure from the normal of a subject's structure, function, or sensation experienced by the subject, but which may not be discoverable simply by examination of the patient. The major categories of signs or symptoms exhibited by a subject with an autoimmune disease may include tissue inflammation, tissue destruction, and pain.

Patients with autoimmune disease often suffer significant inflammation of the kidneys, often causing renal dysfunction and/or destruction, which can lead to death in the absence of dialysis. A clinical laboratory measure of kidney function is creatinine clearance. Creatinine is the metabolic product formed from creatine when food is converted to energy. Creatinine is produced at a steady state and is affected very little by diet or normal physical activities. When a subject's kidneys are damaged and/or cannot function normally, the amount of creatinine excreted in the subject's urine decreases while its level in the blood increases. Thus, the blood level of creatinine indicates how well the kidneys are working. A high level generally means the kidneys are not functioning properly. A creatinine clearance test is performed both on a blood sample taken from the subject's vein and on a sample of urine collected over 24 hours. For example, a blood creatinine level of 3 shows a definite malfunction of the kidneys. A normal creatinine level is less than 1, and normal CrCl levels are typically >100 mL/min. Dialysis is usually required when CrCl levels fall below about 20 mL/min.

Patients with an autoimmune disease may also experience a significant pain, which can be in the joints and the muscles of the back, the esophagus and the bowels. The pain level may come and go in episodes that reduce the normal functioning of an individual. The pain may be independent of the spasms or may correlate with the spasms. Other symptoms that are seen in the autoimmune patients (e.g., rheumatoid arthritis) include the inflammation and tissue destruction of the joints. When this occurs in the hands, the swelling and tissue destruction is sometimes so great that the hands cannot be closed. Another frequent sign in patients with autoimmune disease is the appearance of a “butterfly” rash across the face, particularly in SLE.

While the cause(s) of autoimmune diseases or conditions is/are not known, it is believed that there is/are causative agent(s). The current belief is that each individual condition or disease that is included under the umbrella of “autoimmune disease” probably has a different causative factor or etiology. While not wishing to be bound by any theory of operation of the method of this invention, Applicant's invention is based, at least in part, on the concept that each of the individual conditions included under the classification of autoimmune disease is caused by one or more members of the herpes virus family. This invention is based upon an attempt to stop the production of the antigens stimulating the autoimmune attack. Under the currently accepted medical belief the immune system is behaving abnormally by attacking normal cells and intracellular membranes. The method of this invention is predicated upon the theory that the normal appearing cells (normal to our current technologies) are infected with viral DNA and are producing viral proteins. These viral proteins are “foreign” to the immune system and thus it initiates attack upon them. This results in an “adjuvant effect” for the cell structures. Thus, the immune system begins to attack the normal cell membranes of the nucleus, the mitochondria, the cell wall or other cellular components causing inflammation and tissue destruction. By suppressing the underlying production of “foreign proteins,” there will be no longer “adjuvant” stimulation. Then the immune system may diminish or cease its attack upon the normal membranes.

### Functioning of the Sympathetic Nervous System

In the human body organic and inorganic molecules interact to form organelles and other material to form cells, which in turn combine to form tissue with special functions. Generally these are classified as epithelium tissues, connective tissues, muscle tissue, and neural tissue. Some of these tissues can combine to form organs with various functions that then interact in organ systems. A discussion of these systems and their organization can be found in the tenth edition of “Principles of Anatomy and Physiology” by Tortora and Grabowski, John Wiley and Sons, 2004, Chapters 1-5.

Under normal conditions, all tissues and organs are nourished by an adequate and constant flow of blood through blood vessels to provide the necessary nutrients, transport oxygen and carbon dioxide, remove waste products, and defend against disease. In the normal non-disease state, the flow of blood is regulated by the muscle tension in the walls of the small vessels. This is controlled by the sympathetic division of the autonomic nervous system, i.e., the sympathetic chain or the sympathetic nervous system (“SNS”). The sympathetic chain has billions of nerves in several divisions, each division doing different things. The divisions of the sympathetic nerves serve different end organs. In most organs not all of the small blood vessels are open at the same time. One part of the control by the sympathetic chain is to open and close the various small vessels so that no part of the organ is without adequate blood flow, even though most of the time most of the vessels are essentially closed. When muscles are at rest, not being commanded to contract, a small amount of muscle tension is maintained, by the sympathetic chain, which fires a few nerves at random all the time, so that the muscle does not go flaccid. All muscles, both voluntary and involuntary have a certain “tonus” or “tonicity”, which is the state of normal tension when the muscle is at rest. It is believed that this serves the body by keeping the muscles ready to function in response to a stimulus.

The SNS coordinates cardiovascular, respiratory, digestive, excretory, and reproductive tissue and organ functions, and controls the blood flow to all by control of the wall muscles of the small blood vessels throughout the body. The SNS is often called the “flight or fight” system because it stimulates tissue metabolism, increases alertness, and generally prepares the body to deal with emergencies. Signals from the SNS affect the tonicity of the muscles in the systems. If the SNS is constantly sending excessive rather than normal levels of signals to the blood vessel wall muscles of an organ, the organ may be adversely affected by being chronically denied adequate blood necessary to function normally. Thus organ failure may occur not because of anything other than the sympathetic chain fires too often and the small blood vessels constrict preventing adequate blood flow to the organ, even while the blood pressure is normal. The inability of a muscle to reach the normal resting state is referred to by the inventor as hypertonus or hypertonicity. Hypertonus is defined as the condition where tissues or muscles cannot relax to normal when they are not being commanded to perform a function and should be at rest. This state of hypertonus or increase in resting tone can be any amount of increased tone from very little, to severe unrelenting spasm, or any level in-between. The state of unremitting hypertonicity of the small blood vessel walls can prevent adequate blood flow to the organ, the consequences of which are discussed hereinafter.

Part of the inventor's discovery is that in a human subject exhibiting the signs and/or symptoms associated with reduced blood flow to an organ or tissue, the sign or symptom can be eliminated by administering extraordinarily high levels of an anti-herpetic compound, such as one of Formula (I) or (II), to the subject for a period of time sufficient to increase the blood flow to the tissue or organ. This discovery was unusual in that there is presently no understood explanation as to why the administration of an anti-herpetic compound should cause improved blood flow to a subject's organ or tissue.

While not wanting to be bound by a particular mechanistic theory, the inventor believes that he has developed a viable explanation of his discovery (introduced hereinbefore) and that the explanation leads to an invention that will be defined in its several aspects hereinafter. The inventor believes his data support the conclusion that constant over-contraction of the muscle tissues (hypertonicity) of the arteriolar small blood vessels can be caused by herpetic activity in the sympathetic chain which causes the nerves controlling the arteriolar muscle tone to fire excessively and continuously. This constant over-contraction of the small vessel walls over time will result in the restriction of the flow of blood to the tissue or organ causing it to fail to perform its normal functions in the body. This chronic restriction in blood flow due to hypertonicity of the muscles of the arteriolar wall of the blood vessels to the organs can even damage or destroy the organ.

A subject having a disease correlated with hypertonicity generally will exhibit signs and/or symptoms associated with the disease. The major categories of signs or symptoms exhibited by a subject with a disease associated with abnormally high resting muscle tone (i.e., hypertonicity) may include tissue or organ inflammation, swelling, loss of tissue function, tissue scarring, tissue destruction, and pain.

The blood vessel wall hypertonicity may be induced by the SNS constantly sending neuronal signals to the muscle tissues of the arteriolar blood vessels, thus heightening the normal tonus. It is generally accepted that the SNS is the preferred place in the human body that a herpes virus, e.g., HSV-II, survives for long periods of time. It is also generally accepted that once in the SNS, a herpes virus is never quiescent; it is always reproducing and releasing virus, even if no surface viral vesicles are present. The inventor reasoned that the presence of the virus activity in the nerve cells sufficiently affects the SNS at various segments to modify the signals sent to the target tissue. Excessive signals from the SNS keep the blood vessel wall muscles in a condition of hypertonicity. Thus, the hypertonicity of the muscle tissue is induced by constant herpetic irritation of the SNS, causing the SNS nerves to fire much more than they normally would.

As mentioned hereinbefore, the constant irritation of the SNS is thought to be effected by (1) direct irritation of the cells by the virus or (2) the attack by the subject's immune system on the cells of the sympathetic chain in which the virus reproduces, or (3) both of (1) and (2). The ensuing constant hypertonicity of the muscle of the arteriolar vessels adversely affects the performance of the vessels and significantly reduces the blood flow to an affected tissue or organ, which can result in, amongst other things, inflammation, scarring, destruction, and/or pain in the tissue or organ affected by the reduced blood flow. Where the virus inappropriately tries to create vesicles or just release viral particles on other tissues that are similar to genital tissues, such as joint tissues, the virus releases “foreign viral proteins” on these tissues and the body responds by the immune system attacking the viral proteins. Because of an adjuvant effect, the immune system also attacks normal body tissues in the joints near or adjacent to where the viral particles are released. This attack by the immune system has been accepted by the medical community currently and in the past as the only mechanism which causes all of the damage of autoimmune diseases and these other diseases that often, but not always present with autoimmune diseases.

The ongoing, SNS-induced hypertonus in the arteriolar blood vessels feeding tissues or organs of the human body is an explanation for a host of disease states presently having no prior explanation of their origins. It can thus be seen that certain disease states can be defined as diseases originating from the abnormal functioning of the sympathetic chain, i.e., over-active sympathetic chain syndrome (“OASCS”) or under-active sympathetic chain syndrome (“UASCS”). The diseases can be described as a condition in which the sympathetic chain is over-active and is sending out many more impulses than is normal or is under active and is sending out fewer impulses than is normal. This occurs both when the sympathetic chain is not receiving input and when it is receiving input from its environment. The sympathetic chain has nerves that carry messages into the chain, and in the case of sympathetically-mediated pain, the nerves of the chain forward the pain messages on to the brain. The sympathetic chain controls many body functions that are mostly automatic, i.e., autonomic functions of the body. As mentioned above, these functions include the resting tone of the body tissues, which is called tonus. This inventor has discovered a condition of the sympathetic chain (OASCS), which will not allow tissue such as muscles to relax to their normal resting state (they do not reach normal resting tonus), and a method of treating the condition. The inventor's discovery and further observations lead to an explanation of the origins of a large number of diseases of unknown cause, which often, but not always, present with autoimmune diseases. It has been discovered that treatment and systems in accordance with this invention are more effective than other known treatment attempts and not only stop progression in certain diseases, but also return tissue or organ function to normal where such function has been significantly reduced or lost. The tissues or organs affected maybe those of the eye, salivary glands, nose, heart, lungs, liver, gallbladder, stomach, pancreas, spleen, kidney, bladder, uterus, external genitals, and large and small intestines. No prior treatment ever recovered lost organ function, and the current state of medical knowledge is that organ function lost to this group of diseases, can never be recovered. Until this invention, that was true.

Thus, part of the invention is a new treatment for a large group of previously untreatable diseases, including all autoimmune diseases and other diseases which often present with autoimmune diseases, but not always. The cause of autoimmune disease and these associated diseases is unknown according to the current medical literature. The methods and systems of this invention are based upon a new theory of the cause and mechanisms of these diseases than current medicine proposes. The theory is that both autoimmune diseases and these other diseases are all caused by one or more herpes virus modifying the activity of the SNS, with HSV-II (genital herpes) being one of the worst offenders. As discussed before, it is accepted fact that the herpes virus, especially HSV-II, lives preferentially in the nerve cells of the sympathetic chain. It is also accepted fact that there are many viruses that often are inactive or dormant often for years after an initial infection. However, HSV-II continues to be active, reproducing and releasing virus at a low level, even when there are no obvious signs of virus activity, including herpetic vesicles on the surface of the body of the infected person. This is demonstrated by the fact that many people contact HSV-II even though the person of origin has no vesicles.

Of all the viruses that have widespread distributions in the human population, the herpes viruses display the most aggressive long term continued residency in humans. This can lead to aggressive, long-term testing of the immune system. The herpes family includes Varicella-Zoster (chicken pox-shingles), HSV-I, HSV-II, cytomegalovirus, Epstein-Barr virus and several others that infect humans. When the immune system fails or is suppressed a new presentation of the herpetic vesicle phase commonly results. This is not true for measles, and numerous other common viral illnesses. Herpes displays a preference for and an ability to survive in nervous tissue for long periods. Many of the signs or symptoms of the diseases treated in accordance with this invention are explained by limited focal irritation of the sympathetic nervous system. Herpes viruses display the ability to erupt focally in zoster (shingles) and HSV-I and HSV-II. Animals suffer diseases from this family including Marek's disease in fowl and pseudo-rabies in dogs, cats and cattle. Belief that the herpes virus can cause autoimmune diseases is consistent with the known behavior of the viruses in immune-suppressed individuals. Most children are infected within 5 years of birth and suffer a short episode of varicella (chicken pox). The immune system then suppresses the infection, which then becomes latent only to re-express itself after the age of 50 as shingles, usually very localized presentation involving only one, two or three nerves. This expression almost inevitably occurs in immunosuppressed patients, such as AIDS or transplant patients. HSV-II infects between 25% and 33% of all people in the U.S., according to the CDC. The expression of the most dramatic viral shedding stage of the virus is generally held in check by the immune system, but it represents in immunosuppressed patients, as the viral shedding vesicle eruptions. The detection of the presence or absence or a herpes virus is determined by employing methods known in the art, such as those described in '16207, which is incorporated herein by reference. See pages 20-22 of that document.

The present invention involves administering a compound of Formula (I) or (II), at dosages much higher than previously taught, e.g., the equivalent of famciclovir at doses of at least about 250 mg/kg of body weight per day (and up to about 400 mg/kg/day or more), preferably divided into four about equal doses taken every six hours. Taking the equivalent of, for example, 300-400 mg/kg of famciclovir per day means taking the drug every six hours, i.e., four times in a 24 hour period, so that the total amount taken in the 24-hour period equals 300-400 mg/kg of body weight. It will be recognized from the foregoing discussion that the administration of a compound in accordance with this invention will be for a very long term, in light of the fact that the treatment does not result in the destruction of the virus, but instead minimizes viral shedding to the point where the body's immune system does not react to the presence of the herpes virus. Thus, administration will continue in many cases for the life of the patient.

As discussed before, it was surprising that increased intake of a compound of Formula (I) or (II), e.g., famciclovir at more than 250 mg/kg of body weight per day, the inventor observed significant improvement of impaired kidney function to unexpectedly high levels. The patient's creatinine clearance improved beyond the level where it had remained for at least five years of taking the drug. This high level of restoration is especially remarkable, considering that it was previously thought, that in autoimmune disease, many nephrons are irreversibly destroyed by inflammation. Therefore no restoration of the kidney function was thought to be possible. However, the present invention demonstrates that with high enough amounts of a compound, e.g., one of Formula (I) or (II), the inflammation subsides and kidney function improves to previously unseen levels. Presumably, this very significant improvement of kidney function is due to the many glomeruli being simultaneously in various stages of autoimmune inflammation and destruction, i.e., some of the glomeruli may be inflamed to the point of limited or not functioning, but are then able to resume more normal functioning when their inflammation subsided. This is objective evidence that this technique is superior to previous therapies. It does not just slow the inevitable progression of autoimmune diseases, it stops the inflammation and allows damaged and not destroyed tissue to repair itself with return of function that was previously lost. As the measured kidney function improved a parallel process occurred with a diminution of the visible inflammation, swelling, pain, grinding and clicks in the afflicted articular joints. Renal function tests that evaluate the severity of reduced kidney function can be found in The Merck Manual at pp. 1654-1661. It must be noted that some anti-herpes drugs, because of their poor solubility, have difficulty reaching the cytoplasm of neurons where the virus is acting. The nature of the compound makes effective delivery difficult. If the compounds are modified to make them more hydrophilic, the hydrophilicity creates a problem with respect to nerve cells, which are coated with multiple layers of hydrophobic (fat) membranes. It takes extremely high doses of the anti-herpetic drugs outside the nerve cells in order to achieve significant concentrations inside these cells. To be effective, the drug must penetrate the cells where the viral replication takes place and the viral thymidine kinase, which is the target for the anti-herpes drugs. Thus far, it has been found that famciclovir is the most effective compound in treating the conditions described herein. Thus, the administration of the compounds that are useful in this invention is based on providing a compound to a subject in need thereof at a total daily dose that is equivalent in its effect to at least 250 mg of famciclovir per kg body weight of the subject. It will be recognized that one of skill in the art, such as a medical practitioner or pharmacologist, will recognize how to determine an equivalent effect by administering a compound and monitoring the signs or symptoms of the subject to determine the improvement of the subject's condition. Each subject will react to the administration of a compound useful in the method of this invention in a spectrum of responses, with some subjects requiring more and some less. Thus, subjects may be treated with a compound at the level of 250, 260 270, 280, 290, 300, 310, 320, 330, 340, 350, 360, 370, 380, 390, 400 mg, or more, although it is thought that no more than 400 mg will be needed.

The compounds useful in this invention act by interfering with the action of thymidine kinase in viral replication. Because human thymidine kinase is present in sperm production in male humans, the compounds useful in the method of this invention may have a negative effect (anti-spermatogenic effect) when administered to men at the very high doses set forth. Thus, when administering the compounds, one should consider the risks and benefits of using the process of this invention in treating men. In cases where continued normal functioning is at risk by not taking the drug, damage to the sperm-producing process may well be worth taking a compound in accordance with the teaching of this application. Women, on the other hand, do not have to worry about being administered the high levels of a compound since they do not produce sperm.

From the foregoing, one aspect of the invention is a method treating or preventing a disease in a human subject, where the disease originates from an abnormal functioning of the sympathetic nervous system, which method comprises administering on a daily basis to the subject a therapeutically effective amount of an anti-herpetic compound, e.g., a 2-amino purine derivative such as a compound represented by Formula (I) or Formula (II) below, for a period of time sufficient to alleviate signs or symptoms of the subject associated with the disease. Because the available data correlates the presence of a herpes virus in the subject with the abnormal function of the SNS, the herpes virus is implicated as a causative factor. The therapeutically effective amount of the compound is equivalent in activity to at least about 250 mg famciclovir per kg body weight of the subject per day. It will be recognized by one of skill in the art that famciclovir is a pro-drug for penciclovir, the entity that has activity in the human body. See for example the 2006 edition of the Physician's Desk Reference (PDR) at page 2207. The very high dose treatment is thought to suppress the viral activity in the body. This stops both the production of viral proteins, which is thought to be one cause of the immune system attack on tissues, and allows the sympathetic chain to return to normal function, or firing rates which stops all of the diseases caused by the abnormal functioning of sympathetic chain activity. This in turn results in an improvement in the signs or symptoms of the subject being treated, for example the reduction of pain or inflammation.

Representative 2-amino purine derivatives are approved only for use to treat and suppress herpetic vesicles. For famciclovir (trademark FAMVIR®) the FDA-approved dose may be up to 1500 mg per day for a few days for certain conditions (see the PDR, page 2210). This would amount to about 15 mg/kg per day for a 100 kg person. It is normally given when the tingling occurs which precedes the vesicle formation. This invention is to use doses that are much higher than the recommended dose for the suppression of vesicles. The drug is taken at regular intervals throughout the day, about 3 to 10 times a day to maintain an appropriate, therapeutically effective activity level in the blood stream, not just when “tingles” present. Doses every 6 hours have been found to be useful, e.g., a dose of 7500 mg per dose, 4 times a day. This is 30,000 mg per day or higher for a 100 kg subject. These higher doses are required because these drugs are extremely insoluble in cell membranes, thus very high doses must be ingested for a small but adequate amount to get inside the cells of the sympathetic chain to suppress or significantly reduce activity of virus reproduction, compared to the amount approved by the FDA that is adequate to suppress most herpetic vesicles on the surface.

The possibility of administering standard doses of anti-herpetic drugs to treat autoimmune disease was suggested because a few people taking 2-amino purine derivatives for suppression of vesicles reported that they had some slight relief of autoimmune symptoms. Several investigators attempted to determine if a 2-amino purine would help with autoimmune symptoms. They all reported no benefit in double blind trials, and some reported increased symptoms.

Another unusual aspect of this discovery is that by using the much higher doses than previously taught in '114, the need to titrate a subject to tolerance of the drug could be avoided, that is the higher doses were effective at ameliorating the signs or symptoms of the disease without the accompanying pain of starting at a low dose and increasing it to a therapeutically effective level. While not wishing to be bound by a particular theory, it is thought that by regularly administering the higher levels of the drug, a subject avoids a fluctuating blood level that can add to the irritation of already irritated nerves by major influxes of drug at a high level after having been at a lower level in the subject.

This treatment is far more effective than all prior treatments, including those taught in '114 and '16207, not only in stopping the progression of the diseases, but in returning more of organ function previously lost. Text books claim that organ function lost to these diseases can never be recovered, but this treatment has succeeded in recovering nearly all lost organ function. This success has resulted in an expansion of knowledge as to the mechanisms of damage caused by these diseases. One of the diseases for which a large return of function was achieved was progressive loss of renal function by unknown cause, but suspected to be autoimmune disease related. Text books claim that it is impossible to recover any significant amount of renal function lost to these diseases. This new treatment suggests that the text books are wrong.

These diseases are one of the more common causes of renal failure resulting in dialysis or premature death often in people about age 40, sometimes less. The reason the current thinking appears to be wrong is because the organs do not stop functioning normally because of autoimmune attack upon the cells, as currently believed. The organs stop functioning because the sympathetic chain causes over or under firing levels from these nerves, which are irritated by the herpes virus. And, it is this alteration in firing, i.e., abnormal functioning of the SNS, particularly the increase in firing that is the cause of most of the lost organ function, such as renal failure. This high level of restoration is especially remarkable, considering that it was previously thought, that in autoimmune disease, many nephrons are irreversibly destroyed by inflammation. Therefore no restoration of the kidney function was thought to be possible. However, the present invention demonstrates that with high enough amounts of an anti-herpetic compound, e.g., of Formula (I) or (II), the inflammation subsides and kidney function improves to previously unseen levels. Presumably, this very significant improvement of kidney function is due to the previous lack of flow of blood to the kidneys due to hypertonus of the arteriole vessels feeding them. As a result of reduced blood flow, some of the glomeruli may be inflamed to the point of limited functionality or no functionality at all, but are then able to resume more normal functioning when blood flow increases and their inflammation subsides. This is objective evidence that this technique is superior to previous therapies. It does not just slow the inevitable progression of organ damage, it stops the inflammation and allows damaged and not destroyed tissue to repair itself with return of function that was previously lost. As the measured kidney function improves a parallel process occurs in other affected tissues or organs, with a diminution of the visible inflammation, swelling, pain, grinding and clicks in the afflicted articular joints.

These high dose treatments of this invention suppress the viral activity in the cells of the sympathetic chain, and the firing levels of the sympathetic nerves return to normal. As discussed hereinbefore, the sympathetic chain controls, i.a., the flow of blood to the organs, by control of the muscles of the small blood vessels in the organs. The OASCS can restrict the blood flow to an organ so severely than it cannot function, and eventually result in organ death. The model for this is Raynaud's disease of the hands and feet. The cause of Raynaud's is unknown and there is no effective treatment, according to the medical literature. See The Merck Manual, 17^{th} Edition, page 1790. However, the administration of high doses of anti-herpetic drugs such as 2-amino purine derivative, has successfully returned the blood flow to normal in a subject having HSV-II and Raynaud's disease, and returned renal function to normal, even though it had at one point decreased to 29 mL/min, nearly requiring dialysis for the patient to survive.

The treatment of this invention may be useful for treatment of conditions that are minor or major. For example if the muscles that refuse to relax normally are in the back or leg, the result can be a “charley horse” or an annoying spasm on the peripheral muscles. This might be annoying, but not very dangerous. However, if the area is the sphincter of the outlet to bile duct, billiary inflammation progressing to infection and death can occur. If the area affected is the sphincter to the outlet to the urinary bladder, the condition might have been previously ascribed to Benign Prostatic Hypertrophy (BPH): the outlet refuses to release and the bladder will not empty properly, with the symptoms of hesitancy, small stream, incomplete emptying, and bladder dilatation. The usual treatment for BPH is the removal of some of the Prostatic tissue which is believed in current medical thinking to be preventing the sphincter from relaxing normally. However, it should be noted that this procedure often, and usually does remove some of the bladder sphincter muscle, weakening it. That is one way to relax a muscle in hypertonus, but an extreme technique. By employing a treatment of this invention with a high dose of an anti-herpetic compound, all of the symptoms of BPH are suppressed. This treatment suppresses the OASCS of the nerves controlling the tonus of the bladder sphincter and normal function returns without cutting the muscle to weaken it.

Still another example of improvement that the inventor has discovered is improved pulmonary function. A subject exhibited a pulmonary function measuring about 92% SATS (oxygen saturation) for several years. The pulmonary function was increased to 96% SATS by using the very high dose treatment of this invention. This is a very unusual discovery to see that type of improvement using a drug that does not have a direct action on the lung blood vessel walls. Thus the method of this invention can treat and reverse pulmonary hypertension and pulmonary fibrosis. Although the increase from 92% to 96% O₂ saturation was small, it represents a significant improvement of the matching of blood flow past the alveolar membrane, and improved ventilation of the alveolus. Improvements of this kind in this range with this kind of patient (no known existing lung disease) are unusual. Thus, another aspect of this invention is a method to improve pulmonary function by administering a compound in accordance with the teachings herein. Treatments which cause similar improvements in patients with lung disease are direct acting relaxants upon the muscles of the vessels and the airways of the lung. The compounds of this invention have no such direct effect, and can only relax these critical lung muscles by suppressing the herpetically-induced “hypertonus” or resting tone of these muscles which was elevated above normal tone by the herpetic activity in the cells of the sympathetic chain which control these muscles.

The diseases that are included within the OASCS and UASCS can be organized into seven categories based upon similarity of the conditions caused. All are caused by sympathetic chain over activity except for #7, which is caused by under activity of the sympathetic chain:
1. those involving the pain system of the sympathetic chain;
2. those involving ongoing or chronic hypertonicity of the muscles;
3. those involving sudden muscle spasm-causing hypertonicity;
4. those involving a combination chronic and sudden hypertonicity;
5. those leading to sleep disturbance or restless leg syndrome;
6. those leading to severe fatigue; and
7. those involving the loss of sympathetic nerve function, with loss of control of some autonomic muscles, or loss of sensation, or both.

The following discussion addresses the categories.

### 1. Diseases Relating to the Pain System of the Sympathetic Chain

One of the divisions of the sympathetic chain is an extensive group of nerves which report pain to the brain, the afferent nerves. The over-active sympathetic chain syndrome (OASCS) can involve the afferent pain system of the sympathetic chain, resulting in chronic pain from mild to severe, and hypersensitivity to even light touch which can cause severe pain. Examples include most severe chronic pain syndromes and trigger point syndromes such as fibromyalgia, and severe irritable bowel syndrome, many cases of back pain of unknown origin (e.g., sciatica), dysparunia, and migraine headaches. Tinnitus is another condition in this category. There are many more diseases and pain conditions that are caused by OASCS of the pain nerves of the sympathetic nerve chain, these are just a few examples. Until the recognition of OASCS and the treatment of this invention, there was no effective relief for patients with moderate to severe chronic pain, with trigger points syndrome. The knowledge of even the existence of these disorders is so poor among physicians that often the patients are labeled as malingers, or wanting pain killers to get high, or having some psychiatric problems. This treatment effectively suppresses as much as 95% of the pain, previously not treatable even with high doses of morphine or similar substances. It is possible that Howard Hughes suffered from this condition, which could explain why he spent his last decade lying in bed with no clothes on, and shooting up with morphine. He was not shooting to get high, but to suppress severe pain from the OASCS of the pain nerves. Pain clinics are filled with people seeking relief from these conditions. This treatment is the first to offer significant relief

### 2. Diseases Relating to Chronic Hypertonicity of the Muscles

The sympathetic chain controls the blood flow to all of the tissues and organs of the body by controlling the muscle tension in the muscle walls of the blood vessels which supply the tissues and organs. When the sympathetic chain nerves that control the blood flow fire excessively, the small blood vessels can never relax normally in the affected regions of the body. Essentially what this inventor is proposing is that Raynaud's phenomenon occurs not just in the hands and feet, but in various internal organs. Just as this treatment totally suppresses the cold and pain of hands and feet in Raynaud's, it increases blood flow to various organs, including the kidneys. Just as the hands and feet return to normal color and warmth with the blood flow returned, the kidneys again function when normal blood flow is restored to them. This inventor proposed that Raynaud's and much of the organ function lost of “autoimmune diseases” is actually caused by constant excessive muscle tension (hypertonus), that prevents adequate blood flow to the organ for it to maintain function. If the hypertonus is severe enough the organ may die. As discussed herein, the inventor believes that the excessive firing is caused by the herpetic viruses particularly HSV-II, which lives preferentially in the sympathetic chain nerves, and is always active proven by the production of infectious viruses, even when vesicles are not present. Similar to shingles, one nerve may be highly afflicted causing hypertonus, and the next functioning relatively normally. This selection by nerve(s) is how the hypertonus can be so selective as to afflict only one or two organs. A patient may experience a condition of mild to severe chronic failure of muscles to relax to normal resting tonus when they are not activated by action commands. This condition causes the muscle's resting tone, called tonus, to be heightened and not reach a normal resting tone, i.e., hypertonus of the muscles, but it is one condition of OASCS. The treatment of this invention suppresses OASCS and allows muscles in hypertonus to return to near normal resting tone or tonus. An example is hypertonus of the muscle walls small blood vessels is Raynaud's disease which prevents adequate blood flow to the tissues of the hands and feet. This condition causes severe pain, cold, dark red or blue hands and/or feet, which can progress to tissue death and loss of chunks of the fingers, hands, toes and/or feet. The inventor has demonstrated that this limitation of blood flow similar to Raynaud's disease can and does happen to organs in the body. This is the cause of many different diseases resulting from organ dysfunction and eventual failure to sustain life. Chronic renal failure with or without autoimmune disease is a good example. Another example is the chronic spasm of a coronary artery (Prinzmetal's angina), which may require a stent to keep a segment of a blood vessel wall open where hypertonus occurs. This is a condition often seen in patients having balloon dilation of their coronary arteries. Another example is scoliosis. This is where chronic unrelenting spasm in the paraspineous muscles in children cause the forming bones to be distorted by the unremitting spasm into an S curve. One part of the curve is the area of spasm. The bend of the S in the spine is compensation so that the victim holds their head upright, rather than being bent to the side, without a compensating curve. Severe kyphosis in adults in the metabolic syndrome is caused by severe chronic spasm of the entire thoracic spine muscles which bends the back in a forward curve causing the neck to protrude forward of normal, and the same for the abdominal region. Another condition that can be treated in accordance with this invention is Dupuytren's contracture in which the tendons and muscles of the middle and third fingers cannot relax and thus are forced into a permanently “clawed” position. Another condition is pulmonary hypertension, sometimes referred to as pulmonary fibrosis. Other diseases caused by OASCS will be apparent to one of skill in the art in light of the teachings of this invention.

### 3. Diseases Relating to Sudden Muscle Spasm Syndrome (“SMSS”) Hypertonicity.

The SMSS hypertonicity is part of the OASCS and can lead to spasm of any of the muscles of the body. The muscles suddenly go from relatively normal resting tone to high spasm in an instant, often causing severe pain, and if it is in a critical area it might cause serious secondary effects. The SMSS results in a subject experiencing sudden spasm of any of the muscles of the body. Often this spasm will relent after a few (10) minutes, but sometimes it continues for many minutes (120 or more) before it will suddenly relax to normal. The difference between OASCS caused hypertonus and SMSS is that SMSS causes the muscles to suddenly go from relatively normal resting tone to high spasm in an instant, often causing severe pain. If the spasm is in a critical area it might cause serious secondary effects such as coronary artery contraction. This is one of the causes of what is called a “heart attack.” Some other examples are torta collis, (where the muscles of the neck suddenly spasm twisting the head to one side, and often will not relent for many minutes to hours.), nutcracker esophagus syndrome (where the muscles of the esophagus will suddenly spasm down on the food, and cause extreme pain, usually reverting to normal within 30 minutes), much back pain is caused by SMSS. Another example is sudden emptying of the bladder or bowels without much control.

### 4. Chronic and Sudden Onset Hypertonicity

In some cases, conditions are not easily categorized into either category 2 or 3 show some ongoing or chronic hypertonicity but also some spasm. An example of this would certain gastro-intestinal dysfunction and inappropriate bladder activity, such as sudden emptying of the bowels or bladder. The symptoms in the bladder are often blamed on the increased size of the prostate, often called benign prostatic hypertrophy or BPH, which is discussed hereinbefore.

### 5. Sleep Disturbance and Restless Leg Syndrome

Severe sleep disturbance can be caused by inability to relax the axial muscles due to hypertonus. It is not appreciated by those without hypertonus of the muscles, but it is necessary for the somatic muscles to relax to a predetermined level, before sleep will commence. Hypertonus can prevent that relaxation and prevent sleep. One patient was unable to achieve normal REM sleep for over 15 years, when his autoimmune disease was treated with the current treatments, which are essentially ineffective. However, this same patient was restored to essentially hours of uninterrupted REM sleep on the doses of this invention. Additionally this patient suffered from severe “restless leg syndrome” which is caused by the same disease conditions of the sympathetic chain as causes all above. At least the “restless leg syndrome” is totally suppressed by the treatments of this invention. At the levels of '114 these benefits were only slightly apparent, with the doses of this application normal REM sleep is restored. And, if the drug levels of the 2-aminopurines are allowed to drop for only a few hours the inability to sleep, and the severe restless leg return to the afflicted. When the drug levels are restored by the doses of this application again normal sleep returns, and the “restless leg” is totally suppressed. The relationship of the appearance or suppression of the signs or symptoms of sleep disturbance to the onboard drug level is consistent and dramatic.

### 6. Severe Fatigue

Severe fatigue often cannot be relieved by any current treatment. This is probably a function of OASCS and hypertonus caused by a herpetic infection of the SNS leading to limited blood flow. Being in a constant state of hypertonicity prevents relaxation and rest or sleep, which in turn leads to constant fatigue.

### 7. Loss of Sympathetic Nerve Function

The loss of sympathetic nerve functions, can occur with a loss of control of some function of some autonomic muscles, or loss of sensation or both. This is not as common as 1 through 6, above. And, the effects are usually not as severe. One example is the loss of control of the soft palate, either partial or complete which can cause severe snoring, and is part of the sleep apnea syndrome, and also can cause a snorting sound when talking or laughing, or can cause food to get caught under the soft palate. Another condition is cranial nerve palsies as discussed in the Sixteenth Edition of the Merck Manual at page 2395. Another is acholasia of the esophagus, where the esophagus just does not contract normally, but does not spasm as in nutcracker esophagus, but is essentially flaccid and inactive in the lower half which is controlled by the sympathetic chain. This new treatment effectively suppresses all of these conditions, and in at least one patient return of most lost function of the soft palate has occurred. In another there was significant return of anal sphincter tone with these doses which had been previously lost on the doses of '114.

The inventor has discovered as part of his invention that administration of the anti-herpetic compounds must continue at the desired level even after the signs or symptoms of OASCS have been initially relieved, i.e., if the next dose of this treatment is delayed for more than 6 to 12 hours, the signs or symptoms of the disease conditions return, which will remind the patient when it is time to take the next dose.

The inventor's discovery and observations leads to another aspect of this invention, which is a method for preventing or treating a disease of a tissue or organ originating from an abnormal functioning of a subject's SNS, such as decreased blood flow through blood vessels to a tissue or organ. The method comprises (a) administering an anti-herpetic compound, e.g., Formula (I) or (II), or a pharmaceutically acceptable salt thereof, below, to a human subject having an herpes virus infection, such as HSV-II, for a period of time sufficient to increase the blood flow to the tissue or organ affected and (b) maintaining the administration of the compound to the subject beyond the period of time so that the subject's sympathetic nervous system is normalized, i.e., to achieve a normal tonus of the blood vessels feeding the affected tissue.

Once the intracellular activity of the virus is inhibited by the adequate concentration of a compound such as famciclovir or penciclovir, the irritation of the sympathetic neurons is decreased, and the abnormally increased firing of the sympathetic neurons is diminished. When the sympathetic pain neurons and tonus neurons decrease firing, the pain and the abnormal hypertonus of muscles diminish or disappear. When the smooth muscles relax to normal tonus, the entire group of signs or symptoms caused by their abnormal contraction abates. As a secondary effect, the symptoms caused by diminished blood flow also abate. The pain caused by abnormal firing of the sympathetic neurons, as well as pain caused by tissue hypoxia, also disappears.

It will be appreciated that this invention has two aspects: treatment and prevention. The treatment aspect is directed at conditions that have progressed significantly and organ or tissue functionality has been reduced, with the subject exhibiting advanced signs or symptoms of the condition. The preventative aspect is directed at conditions that have not progressed significantly and organ and tissue function may be slightly reduced, but the subject's signs or symptoms are not as pronounced. Thus, the preventative aspect could be described as a method for preventing the advance of a disease associated with reduced blood to an organ or tissue, which method comprises

(a) examining a human subject for a sign or symptom of the disease,
(b) determining if the patient is infected with a herpes virus, and
(c) if the subject hosts such a virus and exhibits such a sign or symptom, administering a compound of Formula (I) or (II), or pharmaceutically acceptable salt thereof, below, to the subject for a period of time sufficient to prevent the advance of the disease.

Whether the method is to treat or prevent the disease, a compound is administered for a time and at a level that results in amelioration of the signs or symptoms of the disease and allows the subject to function at a normal level. The levels and amount of time that will be needed to achieve results may vary from person to person and can be determined by a physician versed in the arts of administering drugs and evaluating patients. Reduction in signs or symptoms may be seen within one day in some cases but generally a compound will have to be administered for at least a week or more to see positive results. Because the herpetic infection stays dormant in the SNS, the administration will be ongoing at a level that is shown to be effective for a particular individual in most cases for the remainder of a subject's life. The method is useful for both male and female subjects. At very high does, the male sex drive is significantly reduced because of the effect of the compound on the male testes. Females should not see such problems.

If high doses of anti-herpes drugs are administered before the damage and scarring of tissue or organ takes place, particularly the blood vessels supplying these organs, the function of the organs can be returned to near normal when the spasms and hypertonus are relieved.

Another aspect of this invention that flows from the work described hereinbefore is the use of higher amounts of antivirals described herein to prevent prodrome and vesicle outbreaks in a human subject infected with a herpes virus such as HSV-II. The amount of such antivirals needed for such a result is equivalent in activity to about 150 mg of famciclovir per kg body weight of the subject per day. Thus it can be seen that each of the aspects of the invention described hereinbefore apply to preventing prodrome and vesicle outbreak. These include a method of preventing, a system for treating, the use of a compound to prepare a composition, a composition of the compound with an excipient that aids in dissolving or suspending the compound, a liquid composition containing the compound, and the compound combined with another compound that decreases the rate of renal excretion of the antiviral compound. By using a compound equivalent in activity to at least about 300 mg/kg of famciclovir per day, viral shedding is reduced, thus reducing the chance of viral infection spreading between subjects, e.g., by sexual contact for HSV-II.

Another aspect of the invention is a method for prolonging the duration of action of a compound represented by Formula (I) or (II) when administered to a subject having a disease appropriately treated by such a compound. The method comprises co-administering (a) the compound of Formula (I) or (II) with (b) a compound that reduces the rate of renal excretion compound (a) on a daily basis to the subject, while the amount of the compound of Formula (I) or (II) may be at a level presently approved by the FDA, it is preferably equivalent in activity to at least about 50 mg famciclovir per kg body weight of the subject per day. This aspect of the invention is thought to prevent renal crystal growth in a subject receiving an anti-herpetic compound of Formula (I) or (II). This aspect could be also be applied to a system for treating, the use of a compound to prepare a composition, a composition of the compound with an excipient that aids in dissolving of suspending the compound, and a liquid composition containing the compound.

### Compounds Useful in the Invention

The anti-herpetic compounds that are useful for treating diseases in accordance with this invention are exemplified by those represented by Formula (I) and (II) below.

Formula (I) is as follows:

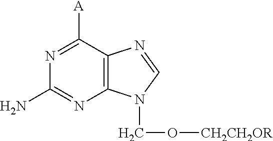

wherein

A is H or OH, and OR is OH, a lower alkyl ester of 2-4 carbon atoms, or OC(O)CH(NH₂)R₄ where R₄ is H or alkyl of 1-4 carbon atoms,

or pharmaceutically acceptable salt thereof.

Representative lower alkyl esters include the acetate, propionate and butyrate esters. Of these the acetate is preferred. The compound that is particularly useful is acyclovir, where A is OH and R is H (which is disclosed in U.S. Pat. No. 4,199,574 issued Apr. 22, 1980 and which is incorporated herein by reference). It should be noted that for both Formulae (I) and (II) where A or B is designated as OH, an alternative representation of the purine ring would be as follows:

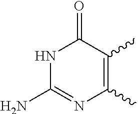

Formula (II) is as follows:

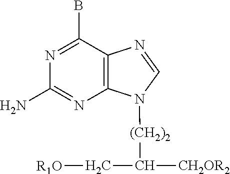

wherein

B is hydrogen, chlorine, alkoxy of 1-6 carbon atoms, phenoxy, phenyl(C₁₋₆)alkyloxy, NH₂, OH or SH, each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group,

or pharmaceutically acceptable salt thereof.

The preferred compounds of Formula (II) are those wherein B is hydrogen, OH or alkoxy of 1-6 carbon atoms (particularly hydrogen or OH) and each of R₁ and R₂ is independently hydrogen or R₃(O), where R₃ is alkyl of 1-6 carbon atoms (e.g., acetyl). Penciclovir and famciclovir are preferred individual compounds, particularly famciclovir.

For purposes of this invention the following definitions are applicable:

Alkyl of 1-6 carbon atoms is a branched or straight chain hydrocarbon of 1-6 carbon atoms represented, e.g., by methyl, ethyl, n-propyl, i-propyl, n-butyl, t-butyl, 1,1-dimethyl-n-propyl, 3-hexyl, and the like.

Alkoxy of 1-6 carbon atoms is a branched or straight chain alkyl attached to an oxygen, i.e., represented by the Formula RO where R is a straight or branched chain alkyl of 1-6 carbon atoms. Representative alkoxy moieties include methoxy, ethoxy, n-propoxy, i-propoxy, n-butoxy, t-butoxy, hexyloxy, and the like.

Aryl includes phenyl which may be optionally substituted with one or two groups selected from alkyl of 1-6 carbon atoms, alkoxy of 1-6 carbon atoms, or halo such as fluoro or chloro.

An acetal is an organic molecule wherein a carbon atom has two single-bonded oxygen atoms attached to it and can be visualized as R—CH—(OR)₂, where the R groups may be the same or different. A cyclic acetal is a molecule wherein the OR groups form a ring with the carbon atom to which they are attached. In a compound of Formula (II), the R₁ and R₂ would be such that a single carbon atom is attached to the two oxygen atoms.

A cyclic phosphate in the context of Formula (II) would be one in which the R₁ and R₂ in the Formula would be a —P(O)OR— to form a ring with the two oxygen atoms shown attached to the R₁ and R₂.

A cyclic carbonate in the context of Formula (II) would be one in which the R₁ and R₂ in the Formula would be a —C(O)— to form a ring with the two oxygen atoms shown attached to the R₁ and R₂.

In the case of compounds of Formula (II) wherein one of R₁ or R₂ is an acyl or phosphate group, the compound exists in two enantiomeric forms. The compounds useful in this invention include both an enantiomers in isolated form and mixtures thereof.

The compounds useful in this invention may be in crystalline form or as a hydrate, and it is intended that both forms are encompassed by the compounds represented by Formulae (I) and (II) above. Examples of pharmaceutically acceptable salts of the compounds of Formulae (I) and (II) above are acid addition salts formed with a pharmaceutically acceptable acid such as hydrochloric acid, orthophosphoric acid and sulfuric acid.

When the compound of Formula (II) contains a phosphate group suitable salts include metal salts, such as aluminum, alkali metal salts such as sodium or potassium, alkaline earth metal salts such as calcium or magnesium and ammonium or substituted ammonium salts, for example those with lower alkylamines such as triethylamine, hydroxy-lower alkylamines such as 2-hydroxyethylamine, bis-(2-hydroxyethyl)-amine or tri-(2-hydroxyethyl)-amine.

Suitable compounds of Formulae (I) and (II) include:
2-amino-9-1,-dihydro-9-[(2-hydroxyethyl)methyl]-6H-purin-6-one (acyclovir)
L-valine, 2-[(2-amino-1,6-dihydro-6-oxo-9H-purin-9-yl)methoxy]ethyl ester, monohydrochloride (valaciclovir hydrochloride);
2-amino-9-(4-hydroxy-3-hydroxymethylbut-1-yl)purine;
2-amino-9-(4-acetoxy-3-hydroxymethylbut-1-yl)purine;
2-amino-9-(4-acetoxy-3-acetoxymethylbut-1-yl)purine (famciclovir);
2-amino-6-oxo-9-(3-hydroxymethyl-4-hydroxybut-1-yl)purine (penciclovir);
2-amino-9-(3-hydroxymethyl-4-methoxycarbonyloxybut-1-yl)purine;
2-amino-9-[2-(2,2-dimethyl-1,3-dioxan-5-yl)ethyl]purine;
2-amino-9-(4-propionyloxy-3-propionyloxymethylbut-1-yl)purine;
2-amino-9-(4-butyryloxy-3-hydroxymethylbut-1-yl)purine;
2-amino-9-(4-benzoyloxy-3-hydroxymethylbut-1-yl)purine;
2-amino-9-(4-hydroxy-3-hydroxymethylbut-1-yl)purine 4′-phosphate;
2-amino-9-(4-hydroxy-3-hydroxymethylbut-1-yl)purine 4′:4″-phosphate;

and pharmaceutically acceptable salts thereof.

The compounds of Formulae (I) and (II), above, are prepared in accordance with the procedures set forth in U.S. Pat. Nos. 4,199,574 issued Apr. 22, 1980; 5,250,688 issued Oct. 5, 1993; 5,246,937, issued Sep. 21, 1993; and 5,075,445 issued Dec. 24, 1991, all of which patents are incorporated herein by reference.

### Administration

For purposes of this invention, it is to be understood that administering a therapeutically effective amount of a compound of Formula (I) or (II), or pharmaceutically acceptable salt thereof, encompasses a method wherein the compound itself is administered via a suitable pharmaceutical composition or a compound that converts into the compound of Formula (I) or (II) upon being administered to subject in need thereof. For example, administering valaciclovir results in plasma levels of acyclovir, which is believed to be the active substance. The administration of famciclovir results in plasma levels of penciclovir, which is believed to be the active substance.

Generally the compounds of Formula (I) or (II), or pharmaceutically acceptable salt thereof, may be administered orally, intramuscularly (IM), intravenously (IV) or parenterally, but because of the ease of oral administration the oral route is generally employed. A composition which may be administered by the oral route to humans may be compounded in the form of a syrup, tablet, caplet, or capsule. When the composition is in the form of a tablet or caplet, any pharmaceutically acceptable excipient suitable for formulating such solid compositions may be used, for example magnesium stearate, starch, lactose, glucose, rice, flour and chalk. The composition may also be in the form of an ingestible capsule, for example of gelatin, to contain the compound, or in the form of a syrup, a solution or a suspension. Suitable liquid pharmaceutical carriers include ethyl alcohol, glycerin, saline and water to which flavoring or coloring agents may be added to form syrups. The compounds may also be presented with a sterile liquid carrier for injection. In general suitable pharmaceutical carriers and methods of preparation can be found in Remington's Pharmaceutical Sciences, 20th Edition.

In general, the type of pharmaceutical excipients and carriers are those that are used in the art for the compositions that already exist. For example, Famvir® is provided as a pharmaceutical composition which contains hydroxypropylcellulose, hydroxypropylmethylcellulose, lactose, magnesium stearate, polyethylene glycol, sodium starch glycolate and titanium dioxide. For acyclovir, Zovirax® capsules contain as an active ingredient acyclovir with inactive ingredients being cornstarch, lactose, magnesium stearate and sodium laurel sulfate, all of which are contained in a capsule shell of gelatin with FD&C Blue No. 2 and titanium dioxide. The tablets of Zovirax® at the 800 mg level contain as inactive ingredients FD&C Blue No. 2, magnesium stearate, microcrystalline cellulose, povidone, and sodium starch glycolate. Zovirax® is also available as a suspension which contains the active ingredient and the inactive ingredients methylparaben, propylparaben, carboxymethylcellulose sodium, flavor, glycerin, microcrystalline cellulose and sorbitol. Valtrex® capsules for oral administration contain valaciclovir hydrochloride and the inactive ingredients, carnauba wax, colloidal silicon dioxide, crospovidone, FD&C Blue No. 2 Lake, hydroxypropylmethylcellulose, magnesium stearate, microcrystalline cellulose, polyethylene glycol, Polysorbate®-80, povidone and titanium dioxide. Other compositions may be apparent to one of skill in the art upon reading this specification.

For intravenous infusion, the compounds may be provided as a sterile powder which is reconstituted with a sterile diluent. For these purposes, for example, in Zovirax® sterile powder, lyophilized acyclovir sodium is used.

Because some subjects treated in accordance with this invention may have difficulty swallowing the number of pills required for effective treatment, it may be advantageous to provide a composition that may be taken as a liquid solution or suspension. This may take the form of a powder or granules that may be mixed with water to form a solution or suspension or it may be a pre-mixed liquid composition. Thus, another aspect of this invention is a composition that comprises (a) a compound of Formula (I) or (II) and (b) a pharmaceutically-acceptable excipient that aids in dissolving or suspending the compound of (a). A liquid composition comprises (a) a compound of Formula (I) or (II) and (b) a liquid pharmaceutical excipient that aids in dissolving or suspending the compound (a).

Very high doses of compounds of Formulae (I) and (II) are needed to provide a therapeutically-effective amount of the compounds. By administering the compounds of this invention at such levels, relief is seen of the signs or symptoms of an autoimmune disease or a disease originating from the abnormal functioning of the SNS, as discussed hereinbefore. While any of the compounds encompassed within the generic formulae of this invention can be used for treating the disease, generally famciclovir is employed because it is more water soluble at body pH of 7.4 than other representative compounds such as acyclovir. The rate and frequency of dosing depends on the extent of the autoimmune conditions, individual tolerance and the particular drug chosen for administration. Generally a therapeutically effective amount is a dosage that is very high relative to the levels effective for the treatment of conditions due to HSV such as HSV I or II or VZV. The therapeutically effective amount administered is sufficient to give the desired blood levels and ultimately the reduction of the signs or symptoms of the condition. The blood levels may vary from individual to individual. To achieve such a blood level, an amount administered on a daily basis will be equivalent to 250 mg or more, e.g., about at least 300, and preferably 300-400 mg famciclovir per kg body weight per day, although the amount administered depends on the activity and bioavailability of the particular compound administered, as well as how an individual responds to the amount administered. In some patients 250 mg/kg/day may be sufficient, while in others more than 300 mg/kg/day will be needed. Thus, for example, amounts such as 250, 260, 270, 280, 290, 300, 310, 320, 330, 340, 350, 360, 370, 380, 390, or 400 mg/kg can be administered. In general the compounds are administered at times throughout a day to maintain a blood level that will continue to ameliorate the autoimmune conditions and provide relief to the individual from the symptoms. Thus the dosing may be equal amounts provided 3 or up to 10 times a day depending on the individual and the compound chosen. Preferably the dosing is 3 to 8 times a day, particularly 4, administered every 6 hours.

In administering famciclovir about 300 to 400 mg/kg/day is employed, e.g., about 30 to 40 g for a 100 kg person. Thus a 50 kg person (i.e., about 110 lbs) would take 15,000 mg to 20,000 of famciclovir per day (50 kg×300 or 400 mg/day), which is at least 25% higher than the previous maximum dosage taught by the '114 patent. While the preferred amount that shows improvement in the case of treating a disease described hereinbefore with famciclovir is about 300 mg/kg/day, the maximum depends on the tolerance of the patient, but is thought to be about 400 mg/kg/day. The following table provides a list of daily minimum/maximum amounts that would be administered to subjects of various weights.

TABLE I

| Weight - kg (lbs) | Dose mg/kg/day | Total Dose/day |
|---|---|---|
| 50 (110) | 250; 300-400 | 12,500; 15,000-20,000 mg |
| 60 (132) | 250; 300-400 | 15,000; 18,000-24,000 mg |
| 70 (154) | 250; 300-400 | 17,500; 21,000-28,000 mg |
| 80 (176) | 250; 300-400 | 20,000; 24,000-32,000 mg |
| 90 (198) | 250; 300-400 | 22,500; 27,000-36,000 mg |
| 100 (220) | 250; 300-400 | 25,000; 30,000-40,000 mg |

It will be appreciated that because of the use of increased dosage of the compounds useful in this invention, an oral pill or tablet will contain larger amounts of a drug compound of Formula (I) or (II). Thus, part of this invention is a container holding a plurality of tablets or capsules, each of which contains 800 to 1200 mg, e.g., at least 1000 mg of the compound of Formula (I) or (II), and wherein the container is associated with a label providing instructions to administer the compound to a subject having a herpes infection and an autoimmune disease or a disease associated with reduced blood flow to a tissue or organ the compound at a level and time sufficient to reduce the signs or symptoms associated with the disease.

In addition to administering an anti-viral compound of Formula (I) or (II) in accordance with this invention, it is useful to also co-administer a compound that decreases the rate of renal excretion of the compound of Formula (I) or (II). Such co-administration may performed by separately administering each compound or by combining the two compounds together in a single composition which may take the form of a capsule, tablet, caplet, syrup elixir, and the like, as discussed hereinbefore. In general the two compounds are administered simultaneously for ease of tracking consumption, but they be administered at different times during the day, if desired. An example of a compound that acts to decrease the rate of renal excretion is probenecid (4-(dipropylsulfamoyl)benzoic acid). The compound is available commercially by the trade name of BENURYL. The normal dosage is suggested to be 500 mg twice per day for a 100 kg person, but may vary from individual to individual. Thus the dosage may vary, for example, from 400 to 600 mg twice per day. If the anti-herpetic drug is administered four times per day, probenecid may be administered four times per day at the rate of 250 mg (e.g., a range of 200 to 300) each time for a 100 kg person. As an example, if a 100 kg person is prescribed 300 mg/kg/day of famciclovir with the dose to be administered in four equal doses every six hours, the person would also be administered a total of about 1000 mg of probenecid. If the two ingredients are combined in an appropriate formulation to provide a tablet containing the appropriate content, such as 1000 mg of famciclovir, probenecid would be present at a level of about 33.33 mg (within a range of about 25 to about 40 mg). Thus the person would ingest 7.5 tablets of the combination product. It will be recognized that some individuals may take slightly more or slightly less or may have a slight variance in the number of tablets taken instead of taking 7.5×1000 mg tablets and still maintain effective blood levels for this invention. For example, a person may have a four times daily schedule such as the following: 1. 7×1000 mg tablets, 2. 8×1000 mg tablets, 3. 7×1000 mg tablets, and 4. 8×1000 mg tablets. Such a regimen provides 30,000 mg per day to a 100 kg person.

Thus, one can see another aspect of this invention, a composition that comprises an anti-viral compound as described hereinbefore, a compound that decreases the renal excretion of the anti-viral compound, and a pharmaceutically acceptable excipient. The composition may be in a dosage form to be orally ingestible, for example in the form of a tablet, caplet, or capsule. The level of the anti-herpetic, when it is famciclovir, is 800 to 1200 mg, while the level of the compound that decreases renal excretion, when it is probenecid, is 25 to 40 mg. Alternatively, the oral composition may be in the form of a liquid for oral administration that is prepared to give the required dosage to achieve the desired level of effectiveness. Again, assuming that the desired daily dosage is 300 mg per day for a 100 kg person, that is 30,000 mg total, a liquid unit dosage is prepared for administration in four equal dosages of about 7500 mg of famciclovir and about 250 mg probenecid. In such a case the composition would be a liquid composition that comprises the anti-herpetic, the renal excretion reducer, and a suitable, pharmaceutically-acceptable liquid to suspend or dissolve one or both compounds to allow for the administration to treat the targeted condition. Another alternative is to provide a dry powder or granular mixture that when mixed with water is suspended or dissolved to provide a liquid composition for easy oral administration. In any of the formulations in which famciclovir and probenecid are combined, the ratio on a weight/weight (w/w) basis may vary from about 25:1 to 50:1, generally about 30:1.

Another important aspect of the present invention is the discovery that the use of very high levels of famciclovir, or an equivalent, results in immediate relief of pain associated with autoimmune disease. The prior art, including the '114 patent, teaches to use pain as guide when adjusting the maximum dose of acyclovir or famciclovir, i.e., “titrating to tolerance.” However, it is now found that a patient could tolerate the pain during the initial period of taking extremely high doses of anti-herpes drugs. After the drugs take effect, usually in less than an hour, the pain is at least 50% reduced or disappears. This is a particularly surprising and useful discovery.

In the past, it was observed, that a patient that stopped increasing the dosage of the anti-herpes drugs at the threshold of pain rarely achieves the relief of the autoimmune signs or symptoms. Patients who exceed the pain threshold and take over 20 g per day (over 200 mg/kg/day) start experiencing a reduction in autoimmune symptoms for prolonged periods of time. The pain in such patients eventually disappears. Patients who take even higher doses, 30 g per day for a 100 kg subject (300 mg/kg) and higher, experience a continuous relief of at least 90% of all of their autoimmune signs or symptoms, including pain, as long as they continue taking the drug.

The following example is given by way of representation of specific administration modes, but should not be interpreted as limiting the scope of the claims in any way.

### Example 1

This example sets forth the treatment of a patient with signs or symptoms of disease associated with reduced blood flow to an organ or tissue (correlated with hypertonicity of the associated blood vessels) using high doses of famciclovir, a compound of Formula (II) where B is H and each of R₁ and R₂ is CH₃C(O)—, at a level and under conditions as explained below.

A patient weighing about 100 kg having multiple signs or symptoms of autoimmune disease had been ingesting famciclovir for several years at about 10 g per day taken 4 times daily during a 24 hour period. While the subject's condition was tolerable, he was in a regular state of pain in the joints and had other signs or symptoms discussed below.

He experienced significant headache pain, which was diagnosed as aseptic meningitis, which was discovered to be treatable initially using 340 mg of famciclovir per kg body weight. This type of meningitis does not involve a bacterial infection, but instead, based on the available case reports, the condition is associated with the presence of HSV-II in the cerebrospinal fluid. The inflammation and pain are therefore thought to be caused by the immune attack on the infected tissues. Within hours of taking the high doses of famciclovir, the patient experienced at least 60% relief of the extremely painful and debilitating symptoms of meningitis. While there have been attempts to treat aseptic meningitis with anti-herpes drugs (see Shalabi et al., “Recurrent benign lymphocytic meningitis”, Clinical Practice, 2006, v. 43, p. 1194), only low doses of acyclovir are suggested. The Shalabi, et al. report is discouraging. According to the authors, the disease is self-resolving. Without a double-blind study, it was not possible to detect the contribution of acyclovir to the recovery. In contrast to such uncertainty, the present inventor observed that unremitting severe symptoms quickly dispersed when famciclovir was taken at a rate of 340 mg/kg famciclovir per day and maintained at 300 mg/kg/day. By comparison, Shalabi et al. use 15-30 mg/kg of acyclovir per day (an equivalent of 3-6 mg/kg of famciclovir per day).

The patient also presented with irritable bowel syndrome (IBS) signs or symptoms, such as severe distention, cramps and pain spasms. After increasing intake of famciclovir to a maintenance level of 300 mg/kg per day, the patient experienced a dramatic improvement. The pain spasms dramatically decreased. The distended waist circumference was reduced from 44 inches to 38 inches over a period of days, and pain was about 90% relieved. Since the patient experienced no weight loss, the reduction in distention was due to the relief of bowel spasms.

The patient also presented signs or symptoms of Raynaud's syndrome, which involves marked or severe vasoconstriction and ischemia in the extremities, usually fingers and toes. As a result, the patient often experienced extreme pain, cold, discoloration, lack of sensation and weakness in the affected parts. The inventor observed that all these signs or symptoms dramatically abated in response to 300 mg/kg of famciclovir per day, taken in four equal doses over a 24 hour period. The signs or symptoms returned when the frequency of taking the drug was lowered from once every 6 hours to once every 10 hours.

The patient also showed the inability to relax and experienced muscle spasms and pain, which led to the inability to sleep. The patient was administered 300 mg/kg of famciclovir per day and experienced relief of 90% of the spasms and pain. As a result, the patient was able to relax and sleep for extended periods of time. The severe bowel and back pain and spasms that the patient experienced were relieved about 80% on this drug regimen.

The same high doses of famciclovir also relieved urination problems in a male patient, such as hesitancy, weak stream and retention. These signs or symptoms are usually associated with benign prostate hypertrophy. However, without taking any prostate-specific drugs, but famciclovir alone, the patient achieved substantial relief of these symptoms. The signs or symptoms disappeared or were at least 90% relieved at 300 mg/kg of famciclovir per day, taken in 6 hour increments. The symptoms returned in about 10 hours when the drug was taken every 10 hours instead of every six hours. When the drug was taken every 6 hours, the symptoms, once again, disappeared.

Another effect of the treatment of this invention was reduction in chest pain. A patient with an autoimmune disease was experiencing severe chest pains or Prinzmetal's angina—a coronary artery spasm and associated cardiac ischemia. After taking high doses of anti-herpes drugs, but no drugs specifically for the heart, the patient reported a marked reduction in the symptoms of Prinzmetal's angina, specifically, 95% reduction in pain at 300 mg/kg of famciclovir per day. In addition, the famciclovir regimen had a beneficial effect on elevated blood pressure. While taking famciclovir at 300 mg/kg per day, the patient was able to reduce his blood pressure lowering medication to a minimum and experience no hypertension.

An improvement in lung function was seen, possibly via relief of pulmonary hypertension, while taking famciclovir at 300 mg/kg per day. The patient experienced a significant improvement in lung function as measured by blood oxygenation (pulse oximetry). The patient's oxyhemoglobin increased from 92% to 96%. This small improvement is significant for this parameter in the 90%-100% range. The patient's pulse O₂ remained at 92% for several years taking about 100 mg/kg of famciclovir, but improved to 96% after six months of taking 300 mg/kg per day of famciclovir.

Yet another effect was alleviation of fibromyalgia: The pain was reduced by about 95% at 300 mg/kg of famciclovir per day.

Another effect of anti-herpes drugs was elimination of the signs or symptoms of rheumatoid arthritis. A patient experienced 100% reduction in joint swelling, pain, inflammation and clicks at 300 mg/kg of famciclovir per day.

A patient was experiencing the loss of control of the soft palate because it could not properly contract, a condition known as cranial nerve palsy. The condition causes snorting during talking and difficulty swallowing food. The food often lodges above the soft palate. These symptoms were 80% eliminated at 300 mg/kg of famciclovir per day. The muscle strength that was absent for over a year, gradually returned over a six-month period after the famciclovir dose was maintained at 300 mg/kg daily.

Another condition observed in a herpes-infected patient was kyphosis or abnormal curvature of the upper spine. Generally, kyphosis is thought to result from habitual poor posture. However, kyphosis is sometimes caused by abnormal contraction of some spinal muscles. When the patient received 300 mg/kg of famciclovir per day, his posture improved as the contracted spinal muscles relaxed.

Another condition experienced by the patient related to muscle spasms referred to as the “nutcracker esophagus.” This condition involves very painful spasms of the esophagus caused by food touching the hypersensitive “trigger points” inside the esophagus. These spasms can persist from minutes to hours. This condition was completely relieved and prevented at 300 mg/kg of famciclovir per day. The condition recurs within a few hours when the drug is stopped.

Additional symptoms relieved by the high doses of anti-herpes drugs involve severe nose and brow acne and rosacea on the chin and lower cheeks. Although the mechanism for these conditions is not understood, they are believed to be of autoimmune origin. Both skin conditions disappeared at 300 mg/kg of famciclovir per day and recurred when the drug was stopped.

Most importantly, the present invention teaches alleviation of pain associated with autoimmune disease. The prior art, including the '114 patent, teaches to use pain as guide when adjusting the maximum dose of the anti-herpes drug. However, it is now disclosed that a patient should tolerate the pain during the initial period of taking extremely high doses of anti-herpes drugs. After the drugs take effect, usually in less than an hour, the pain is at least 50% reduced or disappears.

It was observed, that a patient that stopped increasing the dosage of the anti-herpetic drugs at the threshold of pain rarely achieve the relief of the autoimmune signs or symptoms. Patients who exceed the pain threshold and take over 20 g per day (over 200 mg/kg/day) start experiencing a reduction in autoimmune symptoms for prolonged periods of time. The pain in such patients eventually disappears.

A patient who take even higher doses, 30 g per day for a 100 kg subject (300 mg/kg) and higher, experience a continuous relief of at least 90% of all of their autoimmune signs or symptoms, including pain, as long as they continue taking the drug.

Further aspects of this invention may be apparent to those of skill in the art upon further contemplation of this disclosure.

1. A method for treating or preventing an autoimmune disease or a disease originating from abnormal functioning of the sympathetic nervous system in a human subject, which method comprises administering on a daily basis to the subject a therapeutically effective amount of a compound represented by Formula (I) or Formula (II), or a pharmaceutically acceptable salt thereof, below for a period of time sufficient to alleviate the subject's signs or symptoms associated with the disease, wherein the therapeutically effective amount of the compound is equivalent in activity to at least about 250 mg famciclovir per kg body weight of the subject per day and wherein Formula (I) is 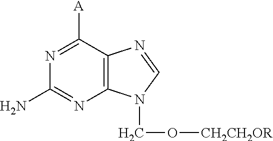
wherein
A is H or OH and OR is OH, a lower alkyl ester of 2-4 carbon atoms, or
OC(O)CH(NH₂)R₄ where R₄ is H or alkyl of 1-4 carbon atoms; and
Formula (II) is 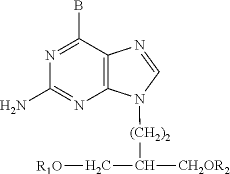
wherein
B is hydrogen, chlorine, alkoxy of 1-6 carbon atoms, phenoxy, phenyl(C₁₋₆)alkyloxy, NH₂, OH or SH;
each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group.

2. The method of claim 1, wherein the compound is famciclovir and the subject is infected with a herpes virus.

3. The method of claim 2, wherein the therapeutically effective amount is about 300 to about 400 mg per kg body weight of the subject per day.

4. The method of claim 1, wherein the compound is famciclovir and is administered to the subject 3 to 10 times a day in equal amounts to maintain a therapeutically effective amount in the blood stream of the subject.

5. The method of claim 4, wherein the compound is administered orally to the subject four times a day every six hours, using a plurality of unit doses, each dose containing 800 to 1200 mg of famciclovir.

6. A method for treatment of a subject exhibiting the signs or symptoms that include chronic pain, failure of muscles to relax, sudden muscle spasm, severe fatigue, or a loss of control of or sensation in autonomic muscle, which method comprises
(a) testing the subject for the presence of a herpes virus;
(b) if the test is positive, choosing a compound of Formula (I) or (II), or a pharmaceutically acceptable salt thereof, that is equivalent in activity against the virus to at least 250 mg of famciclovir per kg body weight of the subject per day;
(c) calculating the amount of the compound needed as therapeutically effective for the subject;
(d) administering the compound at the amount calculated for a period of time sufficient to alleviate the signs or symptoms in the subject; and
(e) continuing the administration of the compound to the subject at the calculated amount for continued alleviation of the subject's signs or symptoms;
wherein
Formula (I) is 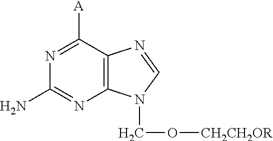
wherein
A is H or OH and OR is OH, a lower alkyl ester of 2-4 carbon atoms, or
OC(O)CH(NH₂)R₄ where R₄ is H or alkyl of 1-4 carbon atoms; and
Formula (II) is 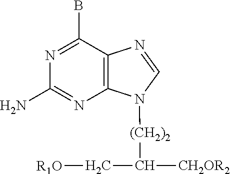
wherein
B is hydrogen, chlorine, alkoxy of 1-6 carbon atoms, phenoxy, phenyl(C₁₋₆)alkyloxy, NH₂, OH or SH;
each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group.

7. The method of claim 6, wherein the compound is famciclovir.

8. The method of claim 7, wherein the therapeutically effective amount is about 300 to about 400 mg per kg body weight of the subject per day.

9. The method of claim 6, wherein the compound is administered to the subject 3 to 10 times a day in equal amounts to maintain a therapeutically effective amount in the blood stream of the subject.

10. The method of claim 9, wherein the compound is administered to the subject four times a day every six hours.

11. A method for treating or preventing a disease in a human subject having signs or symptoms of an autoimmune disease or a disease originating from abnormal functioning of the sympathetic nervous system, which method comprises
(a) testing the subject for the presence of a herpes simplex virus;
(b) if the test is positive, administering on a daily basis to the subject a therapeutically effective amount of a compound represented by Formula (I) or Formula (H), or a pharmaceutically acceptable salt thereof, for a period of time sufficient to alleviate signs or symptoms of the subject associated with the disease, wherein the therapeutically effective amount of the compound is equivalent in activity to at least about 250 mg famciclovir per kg body weight of the subject per day and wherein Formula (I) is 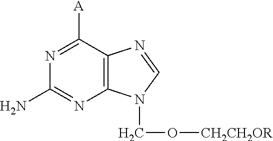
wherein
A is H or OH and OR is OH, a lower alkyl ester of 2-4 carbon atoms, or
OC(O)CH(NH₂)R₄ where R₄ is H or alkyl of 1-4 carbon atoms; and
Formula (II) is 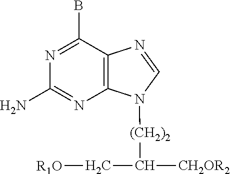
wherein
B is hydrogen, chlorine, alkoxy of 1-6 carbon atoms, phenoxy, phenyl(C₁₋₆)alkyloxy, NH₂, OH or SH;
each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group; and
(c) continuing to administer the compound at a therapeutically effective level to alleviate the signs or symptoms in the subject.

12. The method of claim 11, wherein the compound is famciclovir.

13. The method of claim 12, wherein the therapeutically effective amount is about 300 to about 400 mg per kg body weight of the subject per day.

14. The method of claim 11, wherein the compound is administered to the subject 3 to 10 times a day in equal amounts to maintain a therapeutically effective amount in the blood stream of the subject.

15. The method of claim 14, wherein the compound is administered orally to the subject four times a day every six hours, using a plurality of unit doses, each dose containing 800 to 1200 mg of famciclovir.

16. A method for improving impaired renal function in a human subject having a herpes virus infection, which method comprises administering on a daily basis to the subject a therapeutically effective amount of a compound represented by Formula (I) or Formula (II), or a pharmaceutically acceptable salt thereof, below for a period of time sufficient to improve the impaired renal function of the kidneys in the subject, wherein the therapeutically effective amount of the compound is equivalent in activity to at least about 250 mg famciclovir per kg body weight of the subject per day and wherein Formula (I) is 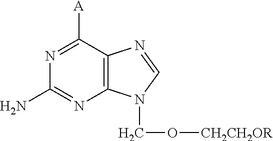
wherein
A is H or OH and OR is OH, a lower alkyl ester of 2-4 carbon atoms, or
OC(O)CH(NH₂)R₄ where R₄ is H or alkyl of 1-4 carbon atoms; and
Formula (II) is 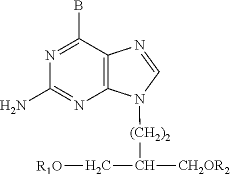
wherein
B is hydrogen, chlorine, alkoxy of 1-6 carbon atoms, phenoxy, phenyl(C₁₋₆)alkyloxy, NH₂, OH or SH;
each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group.

17. The method of claim 16, wherein the compound is famciclovir.

18. The method of claim 17, wherein the therapeutically effective amount is about 300 to about 400 mg per kg body weight of the subject per day.

19. The method of claim 17, wherein the compound is administered to the subject 3 to 10 times a day in equal amounts to maintain a therapeutically effective amount in the blood stream of the subject.

20. The method of claim 19, wherein the compound is administered orally to the subject four times a day every six hours, using a plurality of unit doses, each dose containing 800 to 1200 mg of famciclovir.

21. The method of any of claim 1, wherein a compound that reduces the rate of renal excretion of the compound of Formula (I) or (II) is co-administered to the subject.

22. The method of claim 21, wherein (a) the compound of Formula (I) or (II) is famciclovir and (b) the other compound is probenecid.

23. The method of claim 22, wherein the ratio of compound (a) to compound (b) is in the range of 25:1 to 50:1.

24. A composition for treating a human subject having an autoimmune disease, a disease originating from abnormal functioning of the subject's sympathetic nervous system, or impaired renal function, which composition comprises
(a) a compound, or a pharmaceutically acceptable salt thereof, represented by Formula (I) 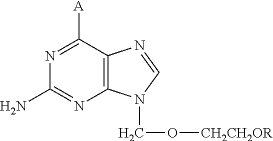
wherein
A is H or OH and OR is OH, a lower alkyl ester of 2-4 carbon atoms, or
OC(O)CH(NH₂)R₄ where R₄ is H or alkyl of 1-4 carbon atoms; or
Formula (II) 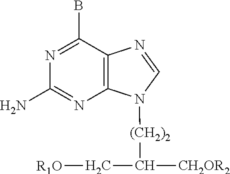
wherein
B is hydrogen, chlorine, alkoxy of 1-6 carbon atoms, phenoxy, phenyl(C₁₋₆)alkyloxy, NH₂, OH or SH;
each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group; and
(b) a pharmaceutically-acceptable excipient that aids in dissolving or suspending the compound of (a) in water so that the pharmaceutical composition may be administered to the subject at a therapeutically effective amount equivalent in activity to at least about 250 mg famciclovir per kg body weight of the subject per day.

25. The composition of claim 24, wherein the compound is famciclovir.

26. The composition of claim 25, wherein the therapeutically effective amount is about 300 to about 400 mg per kg body weight of the subject per day.

27. A liquid composition for treating a human subject having an autoimmune disease or a disease originating from abnormal functioning of the subject's sympathetic nervous system, which composition comprises
(a) a compound, or a pharmaceutically acceptable salt thereof, represented by Formula (I) 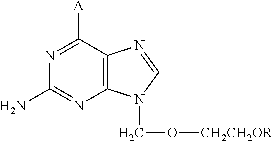
wherein
A is H or OH and OR is OH, a lower alkyl ester of 2-4 carbon atoms, or
OC(O)CH(NH₂)R₄ where R₄ is H or alkyl of 1-4 carbon atoms; or
Formula (H) 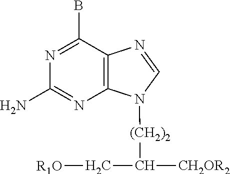
wherein
B is hydrogen, chlorine, alkoxy of 1-6 carbon atoms, phenoxy, phenyl(C₁₋₆)alkyloxy, NH₂, OH or SH;
each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group; and
(b) a liquid pharmaceutically-acceptable excipient that aids in dissolving or suspending the compound of (a) so that the pharmaceutical composition may be administered to the subject at a therapeutically effective amount equivalent in activity to at least about 250 mg famciclovir per kg body weight of the subject per day.

28. The composition of claim 27, wherein the compound is famciclovir.

29. The composition of claim 28, wherein the therapeutically effective amount is about 300 to about 400 mg per kg body weight of the subject per day.

30. The composition of claim 24, wherein a compound that reduces the rate of renal excretion of the compound of Formula (I) or (II) is included in the composition.

31. The composition of claim 30, wherein (a) the compound of Formula (I) or (II) is famciclovir and (b) the other compound is probenecid.

32. The composition of claim 31, wherein the ratio of compound (a) to compound (b) is in the range of 25:1 to 50:1.

33. A composition for treating a human subject having an autoimmune disease or a disease originating from abnormal functioning of the subject's sympathetic nervous system and further being infected with a herpes virus which composition comprises
(a) a compound, or a pharmaceutically acceptable salt thereof, represented by Formula (I) 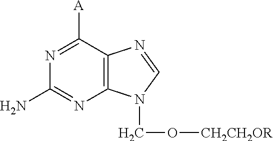
wherein
A is H or OH and OR is OH, a lower alkyl ester of 2-4 carbon atoms, or
OC(O)CH(NH₂)R₄ where R₄ is H or alkyl of 1-4 carbon atoms; or
Formula (II) 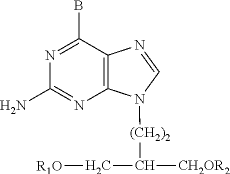
wherein
B is hydrogen, chlorine, alkoxy of 1-6 carbon atoms, phenoxy, phenyl(C₁₋₆)alkyloxy, NH₂, OH or SH,
each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group;
(b) a compound that decreases the rate of renal excretion of the compound of (a); and
(c) a pharmaceutically-acceptable excipient.

34. The composition of claim 33, wherein the compound of (a) is famciclovir and the compound of (b) is probenecid.

35. The composition of claim 34, wherein the w/w ratio of compound (a) to compound (b) is about 25:1 to about 50:1.

36. A composition for treating a human subject having an herpes virus infection, which composition comprises
(a) a compound, or pharmaceutically acceptable salt thereof, represented by Formula (I) 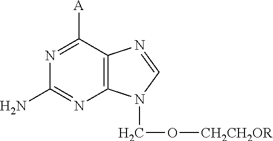
wherein
A is H or OH and OR is OH, a lower alkyl ester of 2-4 carbon atoms, or
OC(O)CH(NH₂)R₄ where R₄ is H or alkyl of 1-4 carbon atoms; or
Formula (II) 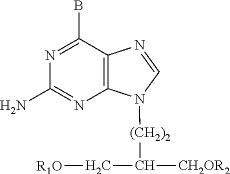
wherein
B is hydrogen, chlorine, alkoxy of 1-6 carbon atoms, phenoxy, phenyl(C₁₋₆)alkyloxy, NH₂, OH or SH;
each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group;
(b) a compound that decreases the rate of renal excretion of the compound of (a); and
(c) a pharmaceutically-acceptable excipient.

37. A product for preventing prodrome and vesicle outbreaks or for reducing viral shedding in a human subject, which product comprises a container holding a composition comprising a compound represented by Formula (I) or Formula (II), or pharmaceutically acceptable salt thereof, below and a pharmaceutically acceptable excipient, wherein the container is associated with instructions for administering the composition to the subject at a daily dose of the compound that is equivalent to at least 150 mg of famciclovir per kg body weight of the subject per day on an ongoing basis to reduce viral shedding, wherein Formula (I) is 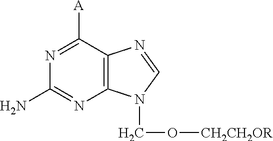
wherein
A is H or OH and OR is OH, a lower alkyl ester of 2-4 carbon atoms, or
OC(O)CH(NH₂)R₄ where R₄ is H or alkyl of 1-4 carbon atoms; and
Formula (II) is 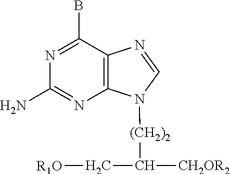
wherein
B is hydrogen, chlorine, alkoxy of 1-6 carbon atoms, phenoxy, phenyl(C₁₋₆)alkyloxy, NH₂, OH or SH;
each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group.

38. A system for treating a human subject having an autoimmune disease, a disease originating from abnormal functioning of the subject's sympathetic nervous system, or impaired renal function, which system comprises
(a) a container holding a pharmaceutical composition comprising a pharmaceutically acceptable excipient and a compound, or a pharmaceutically acceptable salt thereof, represented by Formula (I) 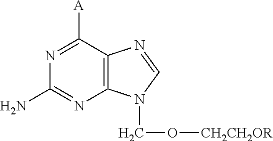
wherein
A is H or OH and OR is OH, a lower alkyl ester of 2-4 carbon atoms, or
OC(O)CH(NH₂)R₄ where R₄ is H or alkyl of 1-4 carbon atoms; or
Formula (II) 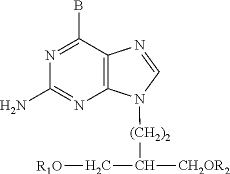
wherein
B is hydrogen, chlorine, alkoxy of 1-6 carbon atoms, phenoxy, phenyl(C₁₋₆)alkyloxy, NH₂, OH or SH,
each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group; and
(b) instructions associated with the container for administering the pharmaceutical composition to the subject at a therapeutically effective amount equivalent in activity to at least about 250 mg per kg body weight of the subject per day.

39. The system of claim 38, wherein the compound is famciclovir and the subject is infected with a herpes virus.

40. The system of claim 39, wherein the therapeutically effective amount is about 300 to about 400 mg per kg body weight of the subject per day.

41. The system of claim 38, wherein the pharmaceutical composition comprising famciclovir and a pharmaceutically-acceptable excipient combined in the form of a plurality of orally-administrable unit dosages each containing 800 to 1200 mg famciclovir per unit is administered to the subject 3 to 10 times a day in equal amounts to maintain a therapeutically effective amount in the blood stream of the subject.

42. The system of claim 41, wherein the compound is administered to the subject four times a day every six hours.

43. The system of claim 38, wherein the instructions provide that a compound that reduces the rate of renal excretion of the compound of Formula (I) or (II) is co-administered to the subject.

44. The system of claim 43, wherein (a) the compound of Formula (I) or (II) is famciclovir and (b) the other compound is probenecid.

45. The system of claim 44, wherein the ratio of compound (a) to compound (b) is in the range of 25:1 to 50:1.

46. A system for treating a human subject having a disease responsive to treatment by a compound of Formula (I) or (II), which system comprises
(a) a container holding a pharmaceutical composition comprising (i) a pharmaceutically acceptable excipient, (ii) an anti-herpetic compound represented by Formula (I) or (II), or pharmaceutically acceptable salt thereof, and (iii) a compound that reduces the rate renal excretion of the anti-herpetic compound, wherein Formula (I) is 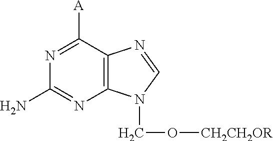
wherein
A is H or OH and OR is OH, a lower alkyl ester of 2-4 carbon atoms, or
OC(O)CH(NH₂)R₄ where R₄ is H or alkyl of 1-4 carbon atoms, and
Formula (II) is 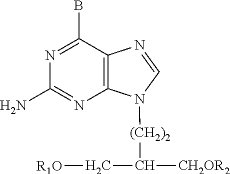
wherein
B is hydrogen, chlorine, alkoxy of 1-6 carbon atoms, phenoxy, phenyl(C₁₋₆)alkyloxy, NH₂, OH or SH,
each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group; and
(b) instructions associated with the container for administering the pharmaceutical composition to the subject at an amount that is therapeutically equivalent in activity to famciclovir of at least about 50 mg per kg body weight of the subject per day.

47. A use of a compound of Formula (I) or (II), or a pharmaceutically acceptable salt thereof, in the preparation of a composition for treating a human subject for an autoimmune disease, a disease originating from abnormal functioning of the sympathetic nervous system, or impaired renal function, wherein Formula (I) is 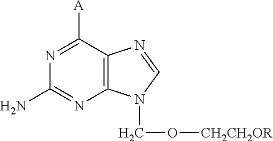
wherein
A is H or OH and OR is OH, a lower alkyl ester of 2-4 carbon atoms, or
OC(O)CH(NH₂)R₄ where R₄ is H or alkyl of 1-4 carbon atoms; and
Formula (II) is 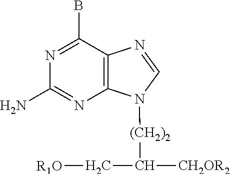
wherein
B is hydrogen, chlorine, alkoxy of 1-6 carbon atoms, phenoxy, phenyl(C₁₋₆)alkyloxy, NH₂, OH or SH;
each of R₁ and R₂ is independently hydrogen, R₃(O) where R₃ is an alkyl of 1-6 carbon atoms or alkoxy of 1-6 carbon atoms, OC(O)CH(NH₂)R₄ where R₄ is H, alkyl of 1-4 carbon atoms, phosphate, or optionally substituted aryl; or R₁ and R₂ are joined together to form a cyclic acetal, a cyclic carbonate or a cyclic phosphate group; and
the composition is administered to the subject at an amount equivalent to the activity in the subject of at least about 250 mg per kg body weight of the subject per day.

48. The use of claim 47, wherein the compound is famciclovir and the subject is infected with a herpes virus.

49. The use of claim 48, wherein the therapeutically effective amount is about 300 to about 400 mg per kg body weight of the subject per day.

50. The use of claim 49, wherein famciclovir is combined with a pharmaceutically acceptable excipient to form an orally-administrable unit dosage form containing 800 to 1200 mg famciclovir per unit, administered to the subject 3 to 10 times a day in equal amounts to maintain a therapeutically effective amount in the blood stream of the subject.

51. The use of claim 50, wherein the compound is administered to the subject in about equal doses four times a day every six hours.
